(19) United States
(12) Patent Application Publication
(10) Pub. No.: US 2021/0378993 A1
(43) Pub. Date: Dec. 9, 2021

(54) TREATMENT OF CYSTINOSIS

(71) Applicant: Nacuity Pharmaceuticals, Inc., Fort Worth, TX (US)

(72) Inventors: G. Michael Wall, Fort Worth, TX (US); Donald Cairns, Aberdeen (SC); Emma Hector, Aberdeen (SC)

(21) Appl. No.: 17/339,652

(22) Filed: Jun. 4, 2021

Related U.S. Application Data
(60) Provisional application No. 63/036,286, filed on Jun. 8, 2020.

(51) Int. Cl. A61K 31/16; A61K 47/55; A61K 31/198; A61K 31/375; A61K 33/00; A61K 33/42; A61K 31/355; A61K 47/24; A61K 31/197; A61K 31/194; A61K 31/085; A61K 31/05; A61K 9/00; A61K 31/145

(52) U.S. Cl. CPC A61K 31/16; A61K 47/55; A61K 31/198; A61K 31/375; A61K 33/00; A61K 33/42; A61K 31/145; A61K 47/24; A61K 31/197; A61K 31/194; A61K 31/085; A61K 31/05; A61K 9/0051; A61K 31/355

(57) ABSTRACT

The present invention relates in general to the field of compositions and methods for using N-acetylcysteine amide (NACA) or (2R,2R′)-3,3′-disulfanediyl bis(2-acetamidopropanamide) (diNACA) for treating cystinosis. This present invention also relates to the general field of using NAC, NACA or diNACA to treat ophthalmic, ocular or corneal effects of cystinosis. This present invention also relates to the general field of using NAC, NACA, diNACA, cysteamine (or cysteamine salts, e.g., the hydrochloride salt or other salts) or any other cystine-depleting agent for treating ophthalmic, ocular or corneal effects of cystinosis by administration in an ophthalmic or ocular insert.

### CROSS-REFERENCE TO RELATED APPLICATIONS

This application claims priority to U.S. Provisional Application Ser. No. 63/036,286, filed Jun. 8, 2020, the entire contents of which are incorporated herein by reference. This application is related to: Wall GM. Prodrug for the treatment of disease and injury of oxidative stress. PCT/US21/14819. Filed 2021 Jan. 23. Wall GM. Methods of Making Deuterium-Enriched N-acetylcysteine Amide (D-NACA) and (2R, 2R′)-3,3′-Disulfanediyl BIS(2-Acetamidopropanamide) (DINACA) and Using D-NACA and DINACA to Treat Diseases Involving Oxidative Stress. AU2018365900B2 (Australia), issue date 2020 Jun. 18. Wall GM. N-acetylcysteine amide (NACA) and (2R,2R′)-3,3′-disulfanediyl bis (2-acetamidopropanamide) (diNACA) for the prevention and treatment of radiation dermatitis and skin lightening, skin whitening and skin improvement. WO2020146666A1, pub 16 Jul. 2020.

### STATEMENT OF FEDERALLY FUNDED RESEARCH

None.

### TECHNICAL FIELD OF THE INVENTION

The present invention relates in general to the field of compositions and methods for using N-acetylcysteine amide (NACA) or (2R,2R′)-3,3′-disulfanediyl bis(2-acetamidopropanamide) (diNACA) for treating cystinosis. This present invention also relates to the general field of using NAC, NACA or diNACA to treat ophthalmic, ocular or corneal effects of cystinosis. This present invention also relates to the general field of using NAC, NACA, diNACA, cysteamine (or cysteamine salts, e.g., the hydrochloride salt or other salts) or other cystine-depleting agents for treating ophthalmic, ocular or corneal effects of cystinosis by administration in an ophthalmic or ocular insert.

### BACKGROUND OF THE INVENTION

Cystinosis is a rare, genetic metabolic disease. Epidemiology. The prevalence of cystinosis is estimated to be 1 in 100,000-200,000 people (Genetic and Rare Disease Information Center Website, rarediseases.info.nih.gov/diseases/6236/cystinosis) Based on a US population of 328,239,523 (US Census Bureau Quick Facts Website, www.census.gov/quickfacts/fact/table/US as of July 1, 2019), this is equivalent to a prevalence of 1641-3282 patients in the US.

Cystinosis is a rare autosomal-recessive lysosomal storage disease that is associated with high morbidity and mortality (Ariceta et al., 2019). It is caused by mutations in the CTNS gene that encodes the cystine transporter, cystinosin, which leads to lysosomal cystine accumulation. There are three clinical forms of cystinosis: infantile or early-onset nephropathic cystinosis, juvenile or late-onset nephropathic cystinosis, and adult (ocular only) cystinosis (Emma et al., 2014). Infantile nephropathic cystinosis is the most severe and most common (about 95% of cases) form of the disease that usually presents within the first year of life.

Nephropathic Cystinosis. In 1903, Abderhalden first described cystine storage disease (Abderhalden, 1903). In people with cystinosis, cystine (an amino acid) builds up in the body's cells and the cells are unable to remove it. When cystine builds up, it forms cystine crystals within cells that can lead to long-term damage to the body's organs—including the kidneys, eyes, liver, muscles, pancreas, and brain. Nephropathic cystinosis is the most severe and most common form of cystinosis, making up 95% of all cases. Nephropathic cystinosis causes severe damage to kidneys and other organs all over the body. Nephropathic cystinosis generally affects boys more than girls and most often occurs in blond-haired, blue-eyed children of European descent. However, people of all races and ethnic backgrounds can be affected. Nephropathic cystinosis symptoms usually appear within a child's first year of life. This damage cannot be reversed, but it can be delayed or reduced (www.procysbi.com/About-Nephropathic-Cystinosis).

Over time, nephropathic cystinosis causes damage to the kidneys. This damage makes the kidneys increasingly unable to absorb essential nutrients and filter out the body's waste—a disorder known as Fanconi syndrome. In people with Fanconi syndrome, nutrients that would normally be absorbed are passed through the kidneys and are eliminated in urine (www.procysbi.com/About-Nephropathic-Cystinosis).

Cystinosis often presents through Fanconi syndrome and leads to cystinosis diagnosis. Cystinosis can be discovered through a white blood cell cystine level test (www.procysbi. com/About-Nephropathic-Cystinosis).

The damage from cystinosis cannot be undone, but it can be delayed or prevented with cysteamine, a cystine-depleting treatment (www.procysbi.com/About-Nephropathic-Cystinosis). The results of treatment with cysteamine vary widely and studies showed that cysteamine improves the outcome of cystinosis but does not provide cure. Hence, over the last decade, cystinosis evolved from a lethal pediatric disorder to a somewhat treatable disorder (David et al, 2019). However, there is a need to find alternative therapeutic strategies.

Ophthalmic, Ocular, or Corneal Cystinosis. The ocular manifestation associated with nephropathic cystinosis was first reported by Burki (Burki, 1941). Corneal cystine crystal accumulation is a common manifestation of all three forms of cystinosis, which overlap to form a continuum of different degrees of severity (Huynh et al., 2013). Corneal changes are the most commonly symptomatic ocular complication in cystinosis (Bishop et al. 2017). In infantile nephropathic cystinosis, cystine crystals are deposited in all layers of the cornea by 16 months of age (Tsilou 2006). In vivo confocal microscopy and anterior segment optical coherence tomography studies reveal that the crystals are predominantly concentrated within the anterior corneal stroma (Emma et al., 2014). Crystals also accumulate in other anterior segment structures, including the conjunctiva, iris, and ciliary body (Tsilou et al., 2002; Bishop 2017). Although early initiation of oral cysteamine therapy, indicated for the treatment of nephropathic cystinosis, can reduce the frequency of posterior-segmental ocular complications, it has no effect on corneal cystine crystals (Tsilou et al., 2006). Corneal crystals diffract incoming light, causing it to scatter, creating photophobia (or light sensitivity), classic to this condition, with severity of photophobia related to density of stromal crystal deposit. Dense corneal stromal changes destabilize the corneal epithelium, resulting in punctate keratopathy, filamentary keratitis, and recurrent epithelial erosions, all of which can cause pain, corneal scarring, and impaired vision (Bishop et al., 2017). Serious complications include severe photophobia (e.g., permanent need to wear sunglasses, reluctance to open eyes in lighted, or even unlighted, room), band keratopathy, recurrent epithelial erosions necessitating corneal transplants (Kaiser-Kupfer et al., 1987; Huynh et al., 2013), and retinopathy (Bishop et al., 2017; Tsilou et al., 2006; Wong et al., 1967).

Pathophysiology of Ophthalmic Effects of Cystinosis. Cystine deposits in the cornea are one of the earliest manifestations of cystinosis, which is also usually present before 1 year of age, but almost always present by 16 months of age (Tsilou et al., 2006; Nesterova and Gahl, 2012). In vivo confocal microscopy and anterior segment optical coherence tomography studies reveal that the crystals are often concentrated within the anterior corneal stroma (Emma et al., 2014). Crystals also accumulate in other anterior segment structures, including the conjunctiva, iris, and ciliary body (Tsilou et al., 2002; Ariceta et al., 2014; Bishop et al., 2017) as well as posterior structures (Tsilou et al., 2006; Wong et al., 1967). In a case report, an 18-year-old boy experienced progressive, severe photophobia, and blepharospasm since age 12 which interfered with his ability to attend school and function in daily life, thereby necessitating corneal transplantation (Kaiser-Kupfer et al., 1987). Analyses of his ocular tissue, besides the expected finding of crystals in corneal stroma, conjunctiva, and iris, also revealed breaks in a thinned Bowman's membrane. These discrete breaks, combined with the deposition of cystine crystals, may contribute to the recurrent corneal erosions, pain and photophobia, which progress in severity and frequency in older nephropathic cystinosis patients. The extrusion of cystine crystals through breaks in Bowman's membrane could cause chronic irritation of the overlying epithelium, at the base of which the afferent sensory nerves to the cornea are located and contribute to excessive ocular pain. In addition, the marked accumulation of iris crystals could contribute to photophobia which can be severe, causing the need to wear sunglasses all day and inability to open eyes in lighted, or even unlighted areas.

Clinical Manifestations of Ophthalmic Effects of Cystinosis. Crystals may be present in the entire peripheral and anterior central stroma. When they are severe, crystals are present throughout the stroma, and the cornea has a hazy, ground glass appearance. Other symptoms may include foreign body sensation, band keratopathy, severe dry eyes, filaments, and limbal neovascularization. Long term complications may include thickening and transillumination of the iris, and development of posterior synechiae, which cause scarring, impeding normal fluid movement, and may lead to angle closure glaucoma and phthisis, formation of a pupillary membrane with crystals (Bishop et al., 2017). Untreated, cystine deposits worsen with time, often resulting in photophobia, ocular discomfort, blurred vision, and in severe cases, band keratopathy and recurrent epithelial erosions, requiring corneal transplants (Kaiser-Kupfer et al., 1987; Huynh et al., 2013).

A study of 208 infantile nephropathic cystinosis patients revealed the most common ocular posterior segment manifestations were pigmentary changes with retinal pigment epithelial mottling, seen as early as infancy. All infantile nephropathic cystinosis patients had significant ophthalmic manifestations involving both the anterior and posterior ocular segment (Tsilou et al., 2006). Incidence of retinopathy (i.e., disease of the retina which results in impairment or loss of vision) was 60% in patients on oral cysteamine therapy for 0-10 years in their cohort of patients [patients with retinopathy-associated symptoms at ages younger than 10 (N=20), 10-19 (N=75), 20-29 (N=47), 30 years and older (N=16)]. Moderate to severe constriction of visual fields, as well as moderate to severe reduction of rod- and cone-mediated electroretinogram (ERG) responses, were observed in older patients. Other less frequently reported eye abnormalities include glaucoma, pigmentary retinopathy, retinal degeneration and optic nerve elevation (Emma et al., 2014). Furthermore, Broyer et al. (1987) described the occurrence of serious problems with ocular function in pediatric cystinosis patients. One patient was almost blind from age 13 years, and at least eight patients were believed to have a reduced ERG response. Also, the optic nerve may be damaged due to papilloedema from raised intracranial pressure.

Retinal degeneration is associated with impaired visual function, as measured by color vision, visual fields, and electrodiagnostic tests (Bishop et al., 2017). Finally, a pigmentary disturbance of the peripheral fundus was seen in eleven of eleven patients with childhood cystinosis (ages 5 weeks to 7.5 years old) examined at the Ophthalmology Branch, National (U.S.) Institute of Neurological Diseases and Blindness. Histologic observations in two of these children demonstrated that extensive degeneration and loss of retinal pigment epithelium was associated with the retinopathy. The peripheral fundus abnormalities may be of special diagnostic value since it can precede the classical corneal deposits of cystine crystals. The existence of this retinopathy appears to be peculiar to childhood cystinosis as it was found to be absent in the adult form of the disease (Wong et al., 1967).

Standard of Care of Ophthalmic Effects of Cystinosis. Specific cystine-depleting treatment with cysteamine currently represents the mainstay of therapy for cystinosis, allowing depletion of lysosomal cystine in most tissues. Although oral cysteamine is not curative, it can improve the overall prognosis and delay progression to end stage renal disease (Emma et al., 2014). However, oral cysteamine therapy does not reach the avascular corneal tissues (Emma et al., 2014). And although corneal transplantation has been performed for disabling ocular symptoms, crystals may reaccumulate after keratoplasty (Huynh et al., 2013).

Currently, the only FDA-approved treatment indicated for the treatment of ocular cystine crystal accumulation in patients with cystinosis is cysteamine hydrochloride eyedrops (CYSTARAN® or CYSTADROPS®) that remove cystine from cells by forming mixed disulfides which can be eliminated. Standard practice is to initiate topical therapy on first evidence of corneal crystals upon eye examination (Bishop et al., 2015). However, cysteamine eyedrops have significant limitations. Cysteamine eye drops are painful (sting) upon instillation and have poor penetration through the corneal epithelium, yielding low concentrations of drug in the stroma, where cystine crystals are vastly deposited (Pescina et al., 2016). In addition, as with most topical drops, cysteamine drops are known to have a brief residence time on the ocular surface, thus requiring repeated (e.g., every waking hour, 12 times per day for CYSTARAN®) administration of treatment, which likely leads to poor compliance (Pescina et al., 2016). The regimen is difficult to achieve for most patients, and those who are unable to adhere to it are encouraged to use CYSTARAN® drops at least six times a day. Furthermore, some patients complain that eyedrops cause a stinging sensation when applied (Pinxten et al., 2017). Cysteamine aqueous solutions are unstable at room temperature as cysteamine oxidizes to its inactive form, cystamine, losing potency and releasing an unpleasant odor in the process (Pinxten et al., 2017). Therefore, it is stored at −20° C. until dispensed, kept refrigerated once opened and has a limited shelf life of 1 week (Huynh et al., 2013). Despite having been demonstrated safe and effective, cysteamine eyedrops cause pain (stinging), exhibit poor efficacy (require dosing every waking hour), are unstable, odiferous, must be kept cold (inconvenient and are discomforting to the eye), and must be discarded 1 week after opening, thereby negatively impacting patients' and caregivers' compliance and quality of life. Further, patients are unable to carry the drops for frequent dosing due to the storage requirements. Even with CYSTARAN® therapy, improvement was observed in only 9% of eyes at 1 year and only up to 30% at 6 years [Summary Basis of Approval NDA 200740; Study STP 869294; 247 patients (mean age 13.8 years)], cystine deposits do not resolve spontaneously, and over time are associated with symptoms of photophobia, recurrent corneal erosions, secondary blepharospasm and loss of visual acuity.

Systemic cysteamine therapy does not reach the avascular corneal tissues, necessitating the use of eyedrops for corneal effects. Despite the availability of these drug products (Elmonem et al.), they all incorporate the same active ingredient, cysteamine, and a need exists for novel agents that are non-toxic that can be used to treat cystinosis.

Drugs marketed in the U.S. for the treatment of cystinosis include the following: CYSTAGON® (1994 US) cysteamine bitartrate 50, 150mg capsules. Indicated for the management of nephropathic cystinosis in children and adults. Adverse events: most common events (>5%) were vomiting 35%, anorexia 31%, fever 22%, diarrhea 16%, lethargy 11%, and rash 7%. Storage: 20° to 25° C. (68° to 77° F.). Protect from light and moisture.

PROCYSBI® (2013 US, Raptor) cysteamine bitartrate delayed-release 25 or 75 mg capsules or packets of 75 or 300 mg granules. Dosage: Adjust dose to achieve a therapeutic target white blood cell (WBC) cystine concentration. Indicated for the treatment of nephropathic cystinosis in adults and pediatric patients 1 year of age and older. Adverse events: Ehlers-Danlos-like Syndrome; skin rash; gastrointestinal (GI) ulcers and Bleeding; CNS Symptoms; leukopenia and/or elevated phosphatase levels; benign intracranial hypertension. Storage: Prior to dispensing, store 2° C. to 8° C. (36° F. to 46° F.); Patient store at 20° C. to 25° C. (68° F. to 77° F.). Protect from light and moisture.

CYSTARAN® (2012 US); mercaptamine HCl solution; dosage=1 drop/eye every waking hour. Indicated for treatment of corneal cystine crystal accumulation in patients with cystinosis. Adverse events: most common adverse reactions (incidence approximately 10% or greater) are sensitivity to light, redness, eye pain/irritation, headache and visual field defects. Storage: −25° C. to −15° C. (−13° F. to 5° F.) until dispensing. Thaw for approximately 24 hours before use. Store thawed bottle at 2° C. to 25° C. (36° F. to 77° F.) for up to 1 week. Do not refreeze. Discard after 1 week of use.

CYSTADROPS® (2020 US, Recordati) (cysteamine ophthalmic solution) 0.37% solution; dosage=1 drop/eye 4 times a day during waking hours. Indicated for the treatment of corneal cystine crystal deposits in adults and children with cystinosis. Adverse events: the most common adverse reactions (≥10%) are eye pain, vision blurred, eye irritation, ocular hyperaemia, instillation site discomfort, eye pruritus, lacrimation increased, and ocular deposits. Storage: Before First Opening: Before opening, store new, unopened CYSTADROPS® in the refrigerator between 36° F. to 46° F. (2° C. to 8° C.). Keep the bottle in the outer carton in order to protect from light. After First Opening: After opening, store opened CYSTADROPS® at room temperature between 68° F. to 77° F. (20° C. to 25° C.). Do not refrigerate after opening. Keep the dropper bottle tightly closed in the outer carton in order to protect from light. Discard 7 days after first opening. (CYSTADROPS® Prescribing Information, 2020).

Finally, certain patents that disclose method for treating cystinosis include: Recordati, oral cysteamine oral U.S. Pat. No. 10,813,888B2; Cysteamine prodrugs, pharmaceutical compositions thereof, and methods of use, U.S. Pat. No. 9,630,917B2; and Methods for storing cysteamine formulations and related methods of treatment, U.S. Pat. No. 10,143,665B2.

Despite these advances, a need remains for novel agents for the treatment of cystinosis that are more bioavailable, effective, stable and/or are safe for daily use, including oral administration. Furthermore, there is a need for novel agents for the treatment of ophthalmic, ocular or corneal cystinosis that are more bioavailable in the eye, effective, stable, do not irritate the eye, and/or are safe for daily use and easy for daily use to increase compliance.

### SUMMARY OF THE INVENTION

In accordance with an embodiment, the present invention provides a method for the treatment of cystinosis in an animal or human that comprises administering to the animal or human a therapeutically effective amount of N-acetylcysteine amide (NACA) or (2R,2R′)-3,3′-disulfanediyl bis(2-acetamidopropanamide) (diNACA) sufficient to treat, or reduce the symptoms of, cystinosis. This present invention also provides a method for the treatment of ophthalmic effects of cystinosis in an animal or human that comprises administering to the animal or human a therapeutically effective amount of NAC, NACA or diNACA to treat ophthalmic, ocular or corneal effects of cystinosis. This present invention also provides a method for the treatment of ophthalmic effects of cystinosis in an animal or human that comprises administering to the animal or human a therapeutically effective amount of NAC, NACA, diNACA or cysteamine (or cysteamine salts, e.g., the hydrochloride salt or other salts) by administration in an ophthalmic or ocular insert containing NAC, NACA, diNACA or cysteamine (or cysteamine salts, e.g., the hydrochloride salt or other salts). NACA (NPI-001) and diNACA (NPI-002) were compared to cysteamine with regard to lack of cytotoxicity in human cystinotic cell culture and depletion of cystine in human cystinotic cell culture.

Surprisingly, in the present studies of cell viability in human cystinotic cell culture, NACA (NPI-001) at all concentrations was statistically superior to cysteamine at the same concentrations with regard to increasing human cystinotic cell viability after 72 hours (FIG. 2). Also surprisingly, diNACA (NPI-002) at the lowest concentration, 25 μM, was statistically superior to cysteamine at the same concentration, with regard to increasing human cystinotic cell viability after 48 or 72 hours (FIG. 1 and FIG. 2).

Surprisingly, in the present studies of cystine-depleting activity in human cystinotic cell culture, NACA was superior to cysteamine and after 6, 24, 48 and 72 hours (FIGS. 3-6). Also, surprisingly, diNACA was superior to cysteamine after 6, 24 and 48 hours (FIGS. 3-5). Also, surprisingly, NACA was statistically superior to cysteamine after 6, 24 and 48 hours (FIGS. 3-5). Also, surprisingly, diNACA was statistically superior to cysteamine after 6 and 48 hours (FIGS. 3 and 5).

N-acetylcysteine (NAC) is a thiol-containing antioxidant that has been approved by the U.S. FDA as an antidote for hepatotoxicity caused by acetaminophen overdose and used for over 50 years for clinical applications, including mucolytic therapy for respiratory conditions with excessive and/or thick mucus production and others (Kelly, 1998). Oral NAC has also been shown to improve cystinosis, presumably through its antioxidative effects to reduce oxidative stress (Guimaraes et al., 2013; Vaisbich et al, 2011). In a 3-month study of cystinosis patients without renal replacement therapy, oral cysteamine plus oral NAC reduced oxidative stress and significantly improved renal function (Guimaraes et al, 2013). However, use of NAC has limitations, including low membrane penetration, low systemic (Ates et al, 2008, Kahns and Bundergaard, 1990) bioavailability. N-acetylcysteine amide (NACA; NPI-001) has greater lipophilicity and, therefore, greater cell permeability than NAC, and should offer improved efficacy in certain disorders (Sunitha et al, 2013, Atlas et al, 1999). The major metabolite of NACA is NAC. Since NAC has been shown efficacious in cystinosis, NACA (a lipophilic prodrug of NAC) should also be efficacious.

NACA contains a primary thiol group like cysteamine which is indicated for the oral and ocular treatment of cystinosis. The primary thiol NACA should, like cysteamine, react with cystine to form a mixed disulfide. Evidence includes the fact that during the development of analytical methods for total NACA in tissues, a reagent was needed to reduce NACA-mixed disulfides that spontaneously formed in tissue (King et al., 2019).

diNACA is a prodrug of NACA and NAC (PCT/US21/14819).

Other known anticystinosis agents have chemical structural similarity to, yet distinct from NACA and diNACA (Buchan et al., 2010; Buchan et al., 2012; McCaughan et al., 2008; Omran et al., 2011a; Omran et al., 2011b; Omran et al., 2011c).

Elevated levels of thiobarbituric acid-reactive substances (TBARS), biomarkers of lipid peroxidation (oxidative stress), have been found in the sera of cystinosis patients (Vaisbich 2011; Guimaraes et al., 2013); this observation suggests that increased oxidative stress plays a role in the pathogenesis of the cystinosis. NACA reduces oxidative stress in numerous diseases (Sunitha et al., 2013) and so should also reduce oxidative stress in cystinosis. Similarly, diNACA had been shown to reduce oxidative stress in tissue (PCT/US21/14819) and so should also reduce oxidative stress in cystinosis. NACA and diNACA protects skin cell cultures from oxidative stress conditions (Neil et al., 2020) and so should also reduce oxidative stress in cystinosis.

Other research has shown that NACA protects retinal pigmented epithelial (RPE) cells cultures from oxidative stress conditions (Schimel et al., 2011).

In one aspect, the NACA or diNACA is provided in or with a pharmaceutically acceptable carrier. In another aspect, the NACA or diNACA is administered intraocularly, subretinally, intravitreally, posterior juxtasclerally, orally, intravenously, intramuscularly, topically, sublingually, topical ocular, ocular implant, via ocular insert or rectally. In another aspect, the NACA or diNACA is administered in daily doses of about 0.5 to 150 mg/Kg. In another aspect, NACA or diNACA is administered two or three times daily. In another aspect, NACA or diNACA is administered with a second active agent selected from at least one of ascorbic acid, cysteamine (any salt form), cysteine hydrochloride, sodium bisulfate, sodium metabisulfite, sodium sulfite, ascorbyl palmitate, butylated hydroxyanisole (BHA), butylated hydroxytouene (BHT), lecithin, propyl gallate, α-tocopherol, citric acid, ethylenediamine tetraacetic acid (EDTA), sorbitol, tartaric acid, or phosphoric acid. In another aspect, the dose for administration is 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 milligrams per dose. In another aspect, the NACA or diNACA is delivered orally via a mini-tablet, capsule, tablet, effervescent, dual release, mixed release, sachet, powder, ophthalmic or ocular insert, eyedrop, ocular implant or liquid. In another aspect, the animal is a human.

In accordance with another embodiment, the present invention includes a method for the treatment of ophthalmic effects of cystinosis with NAC or cysteamine (any salt form) administered in an ophthalmic or ocular insert. In another aspect, the NAC or cysteamine (any salt form) is administered in daily doses of about 0.5 to 150 mg/Kg. In another aspect, NAC or cysteamine (any salt form) is administered two or three times daily. In another aspect, NAC or cysteamine (any salt form) is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytouene (BHT), lecithin, NACA, diNACA, propyl gallate, α-tocopherol, citric acid, ethylenediamine tetraacetic acid (EDTA), sorbitol, tartaric acid, or phosphoric acid. In another aspect, the dose for administration is 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose.

### BRIEF DESCRIPTION OF THE DRAWINGS

For a more complete understanding of the features and advantages of the present invention, reference is now made to the detailed description of the invention along with the accompanying figures and in which:

FIG. 1 shows human cystinotic fibroblast cell viability after exposure to 25, 50 or 75 μM cysteamine, NACA (NPI-001), diNACA (NPI-002), respectively, versus no treatment, for 48 hours.

FIG. 2 shows human cystinotic fibroblast cell viability after exposure to 25, 50 or 75 μM cysteamine, NACA (NPI-001), diNACA (NPI-002), respectively, versus no treatment, for 72 hours.

FIG. 3 shows relative cystine concentration as percent of control in human cystinotic fibroblast cell culture after exposure to 50 μM cysteamine, NACA (NPI-001) or diNACA (NPI-002) for 6 hours.

FIG. 4 shows relative cystine concentration as percent of control in human cystinotic fibroblast cell culture after exposure to 50 μM cysteamine, NACA (NPI-001) or diNACA (NPI-002) for 24 hours.

FIG. 5 shows relative cystine concentration as percent of control in human cystinotic fibroblast cell culture after exposure to 50 μM cysteamine, NACA (NPI-001) or diNACA (NPI-002) for 48 hours.

FIG. 6 shows relative cystine concentration as percent of control in human cystinotic fibroblast cell culture after exposure to 50 μM cysteamine, NACA (NPI-001) or diNACA (NPI-002) for 72 hours.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

N-acetylcysteine (NAC) has the chemical structure:

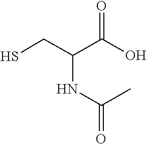

N-acetyl-L-cysteine amide (NACA), also known as (R)-2-(acetylamino)-3-mercapto-propanamide, N-acetyl-L-cysteinamide, or acetylcysteinamide, has the chemical structure:

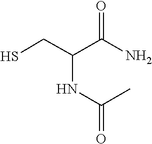

N-acetylcysteine amide (NACA), the amide form of N-acetyl-L-cysteine (NAC), acts as a carrier or prodrug to NAC.

(2R,2R′)-3,3′-disulfanediyl bis(2-acetamidopropanamide) (diNACA), has the chemical structure:

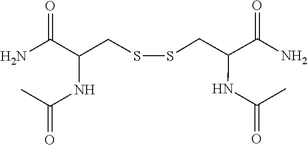 [SMILES: CC(=O)NC(CSSCC(NC(C)=O)C(N)=O)C(N)=O]

(2R,2R′)-3,3′-disulfanediyl bis(2-acetamidopropanamide) (diNACA), the dimer form of N-acetyl-L-cysteineamide, acts as a carrier or prodrug to NAC or cysteine. NAC, NACA and diNACA increase levels of Gluthathione (GSH). GSH is a tripeptide, c-L-glutamyl-L-cysteinyl-glycine, found in all mammalian tissues. It has several important functions including detoxification of electrophiles, scavenging reactive oxygen species (ROS), maintaining the thiol status of proteins, and regeneration of the reduced forms of vitamins C and E. GSH is the dominant non-protein thiol in mammalian cells; as such it is essential in maintaining the intracellular redox balance and the essential thiol status of proteins. Also, it is necessary for the function of some antioxidant enzymes such as the glutathione peroxidases.

Intracellular GSH levels are determined by the balance between production and loss. Production results from de novo synthesis and regeneration of GSH from GSSG by GSSG reductase. Generally there is sufficient capacity in the GSSG reductase system to maintain all intracellular GSH in the reduced state, so little can be gained by ramping up that pathway. The major source of loss of intracellular GSH is transport out of cells. Intracellular GSH levels range from 1-8 mM while extracellular levels are only a few μM; this large concentration gradient essentially precludes transport of GSH into cells and once it is transported out of cells, it is rapidly degraded by γ-glutamyltranspeptidase. Inhibition of GSH transporters could theoretically increase intracellular GSH levels, but is potentially problematic because the transporters are not specific for GSH and their suppression could lead to an imbalance of other amino acids and peptides. Thus, intracellular GSH levels are modulated primarily by changes in synthesis.

GSH is synthesized in the cytosol of virtually all cells by two ATP-requiring enzymatic steps: L-glutamate+L-cysteine+ATP[→]γ-glutamyl-L-cysteine+ADP+Pi and γ-glutamyl-L-cysteine+L-glycine+ATP [→]GSH+ADP+Pi. The first reaction is rate-limiting and is catalyzed by glutamate cysteine ligase (GCL, EC 6.3.2.2). GCL is composed of a 73 Kd heavy catalytic subunit (GCLC) and a 30 Kd modifier subunit (GCLM), which are encoded by different genes. GCCL is regulated by nonallosteric competitive inhibition of GSH (Ki=2.3 mM) and by the availability of L-cysteine. The apparent Km of GLC for glutamate is 1.8 mM and intracellular glutamate concentration is roughly 10-fold higher so that glutamate is not limiting, but the Km for cysteine is 0.1-0.3 mM, which approximates its intracellular concentration. The second reaction is catalyzed by GSH synthase (GS, EC 6.3.2.3), which is 118 Kd and composed of two identical subunits. While GS is not felt to be important in regulation of GSH synthesis under normal conditions, it may play a role under stressful conditions because in response to surgical trauma, GSH levels and GS activity were reduced while GCL activity was unchanged. Furthermore, compared to increased expression of GCLC alone, increased expression of both GCLC and GS resulted in higher levels of GSH. In order to maximize the effects of increasing synthetic enzymes, it is necessary to provide increased levels of cysteine. In cultured neurons, 90% of cysteine uptake occurs through by the sodium-dependent excitatory amino acid transporter (EAAT) system. There are five EAATs and cysteine uptake by neurons occurs predominantly by EAAT3, more commonly known as excitatory amino acid carrier-1 (EAAC1). Under normal circumstances most EAAC1 is in the endoplasmic reticulum (ER) and only translocates to the plasma membrane when activated. This translocation is negatively regulated by glutamate transporter associated protein 3-18 (GTRAP3-18) and suppression of GTRAP3-18 increased GSH levels in neurons. Thus, internalization of cysteine provides a roadblock for GSH synthesis, but fortunately it can be bypassed by N-acetylcysteine (NAC), which readily enters cells even in the absence of activated EAAC1. Systemically administered NAC gains access to the CNS, increases GSH levels, and provides benefit in neurodegenerative disorders in which oxidative stress is an important part of the pathogenesis.

All cellular compartments must be protected against oxidative damage, including the cytoplasm, mitochondria and the nucleus. The present inventors have previously performed gene transfer of enzymes that detoxify reactive oxygen species, but that approach requires expression of two enzymes in the cytoplasm and two enzymes in mitochondria. In contrast, the present invention provides for protection of all cellular compartments with expression of only two enzymes in the cytosol because GSH is able to diffuse everywhere throughout cells.

NAC is used for the treatment of acetaminophen overdose with 140 mg/kg as the loading dose, followed by 70 mg/kg every 4 hours for 17 doses, starting 4 hours after the loading dose. In clinical studies, NAC has been administered orally from 400 to 1000 mg once daily and from 200 to 600 mg three times daily. However, following an oral dose of 600 mg in humans, NAC is rapidly absorbed and then rapidly cleared. The plasma half-life of NAC has been reported to be 2.5 hours and no NAC is detectable 10-12 hours after administration. During absorption, NAC is rapidly metabolized to cysteine, which is a direct precursor of glutathione. diNACA is a prodrug for NACA and NAC, and NACA is a prodrug for NAC (PCT/US21/14819).

Ophthalmic Drop (Eyedrops) and Ointment Formulations. Eye drops are typically aqueous or aqueous and oil solutions, emulsions, or suspensions of one or more active ingredients, which may contain preservatives if stored in multiuse packaging. Eye formulations are sterile and isotonic. The optimum pH for eye drops equals that of tear fluid, about pH 7.4. The stability of active ingredient and the tissue tolerance to the preparation will dictate the requirement for buffer. If the pH exceeds pH 4 to 8, the formulation may cause discomfort and/or irritation (e.g., burning, stinging), and drug bioavailability can decrease because of increased tearing (Barnaowski et al., 2014). Components may include buffers such as citrate, phosphate or borate, preservatives such as mercuric salts, polyquaternium, zinc salts, and benzalkonium salts, lubricants such as glycerin, surfactants such as tyloxapol, and polysorbates, viscosity modifiers such as hydrophilic polymers of high molecular weight which do not diffuse through biological membranes and which form three-dimensional networks in the water such as polyvinyl alcohol, poloxamers, hyaluronic acid, carbomers, and polysaccharides, such as, cellulose derivatives, gellan gum, and xanthan gum, carriers such as polyoxyethylene-polyoxypropylene block copolymer (poloxamer 407), mucoadhesive hydrophilic polymers such as macromolecular hydrocolloids with many hydrophilic groups (carboxyl, hydroxyl, amide, and sulfate), polyacrylic acid, sodium carboxymethyl cellulose, and chitosan, as well as lectins, cross-linked polyacrylic acids which exhibit mucoadhesive properties, such as carbomer and carbopol, and cyclodextrins (Barnaowski et al., 2014).

Ointments usually contain solid or semisolid hydrocarbon base of melting or softening point close to body temperature (Barnaowski et al., 2014).

Carriers may include liposomes built of phosphatidylcholine, stearylamine, and various amounts of cholesterol or lecithin and α-L-dipalmitoylphosphatidylcholine, SolulanC24, a derivative of lanolin, which is a mixture of ethoxylated cholesterol (ether of cholesterol and polyethylene glycol), ethoxylated fatty alcohols (ether of cetyl alcohol and polyethylene glycol), and polyamidoamine (PAMAM) (Barnaowski et al., 2014).

The eye drop dosage form is easy to install but suffers from the inherent drawback that most of the instilled volume is eliminated from the pre-corneal area resulting in a bioavailability ranging from 1-10% of total administrated dose. The poor bioavailability and rapid pre-corneal elimination of drugs given in eye drops is mainly due to conjunctival absorption, rapid solution drainage by gravity, induced lachrymation, blinking reflex, low corneal permeability and normal tear turnover. Because of poor ocular bioavailability, many ocular drugs are applied in high concentrations. This cause both ocular and systemic side-effects, which is often related to high peak drug concentrations in the eye and in systemic circulation. The frequent periodic instillations of eye drops are necessary to maintain a continuous sustained therapeutic drug level. This may result in a massive and unpredictable dose of medication (Rathore and Nema, 2009).

Suspension types of pharmaceutical dosage forms are formulated with relatively water insoluble drugs to avoid the intolerably high toxicity created by saturated solutions of water-soluble drugs. However, the rate of drug release from the suspension is dependent upon the rate of dissolution of the drug particles in the medium, which varies constantly in its composition with the constant inflow and outflow of lachrymal fluid. In order to overcome the limitations of (a) short residence time, (b) pulsed dosing of drug, (c) frequent instillation, (d) large drainage factor, other delivery methods may be employed, including ophthalmic inserts (Rathore and Nema, 2009).

Ophthalmic Inserts. Ophthalmic inserts are sterile, thin, multilayered, drug-impregnated, solid or semisolid devices placed into cul-de-sac or conjunctival sac and whose size and shape are especially designed for ophthalmic application. They are composed of a polymeric support containing drug(s) incorporated as dispersion or a solution in the polymeric matrix. The main objective of an ophthalmic insert is to increase the contact time between the preparation and the conjunctival tissue to ensure a sustained release to the ocular surface. In comparison with the traditional ophthalmic preparation i.e., eye drops, solid ophthalmic inserts may offer advantages such as (a) increased contact time and bioavailability, (b) prolonged drug release and thus better efficacy, (c) reduction of adverse effects, and (d) reduction of the number of administrations and thus better patient compliance. The foreign-body sensation of an insert presents a challenge. Discomfort leads to poor-patient compliance, excessive lachrymation that accompanies irritation, dilutes the drug and causes reduction in its concentration. A properly designed ocular insert will minimize the sensation caused by its insertion and wear. Desired criteria for a controlled release ocular insert include: (1) Ease of handling and insertion; (2) Lack of expulsion during wear; (3) Reproducibility of release kinetics (e.g., zero-order drug delivery); (4) Applicability to variety of drugs; (5) Non-interference with vision and oxygen permeability; (6) Sterility; (7) Stability; and/or (8) Ease of manufacture Classification of patented ocular inserts (Rathore and Nema, 2009).

Diffusion-based inserts. Diffusion inserts are composed of a central reservoir of drug enclosed in specially designed semi-permeable or micro porous membranes, which allow the drug to diffuse the reservoir at a precisely determined rate. The drug release from such a system is controlled by the lachrymal fluid permeating through the membrane until a sufficient internal pressure is reached to drive the drug out of the reservoir. The drug delivery rate is controlled by diffusion through the membrane. The central reservoir may be composed of glycerin, ethylene glycol, propylene glycol, water, methyl cellulose mixed with water, sodium alginate, poly (vinylpyrrolidone) or polyoxyethylene stearate. Membranes may be composed of polycarbonates, polyvinyl chloride, polysulfones, cellulose esters, crosslinked poly (ethyl oxide), cross-linked polyvinylpyrrolidone, and cross-linked polyvinyl alcohol (Rathore and Nema, 2009). Copolymers for minidiscs may include α-ω-bis(4-methacryloxy)-butyl poly(dimethylsiloxane) and poly(hydroxyethyl methacrylate) (Barnaowski et al., 2014).

Osmotic inserts. Osmotic inserts are generally composed of a central part surrounded by a peripheral part. The central part may be composed of a single reservoir or two distinct compartments. In first case, it is composed of a drug with or without an additional osmotic solute dispersed through a polymeric matrix, so that the drug is surrounded by the polymer as discrete small deposits. In the second case, the drug and the osmotic solutes are placed in two separate compartments, the drug reservoir being surrounded by an elastic impermeable membrane and the osmotic solute reservoir by a semi-permeable membrane. The second peripheral part of osmotic inserts comprises in all cases a covering film made of an insoluble semi-permeable polymer. Tear fluid diffuses into peripheral deposits through the semi-permeable polymeric membrane, wets and induces dissolution. The solubilized deposits generate a hydrostatic pressure against the polymer matrix causing its rupture under the form of apertures. Drug is then released through these apertures from the deposits near the surface of the device, which is against the eye, by the sole hydrostatic pressure. This corresponds to the osmotic part characterized by zero order drug release profile. Water permeable matrices may include ethylene-vinyl esters, copolymers, plasticized polyvinyl chloride (PVC), polyethylene and cross-linked polyvinylpyrrolidone (PVP). Semi-permeable membranes may include cellulose acetate derivatives, ethyl vinyl acetate (EVA), or polyesters of acrylic and methacrylic acids (Eudragit®). Osmotic agents may include inorganic components such as magnesium sulfate, sodium chloride, potassium phosphate, sodium carbonate and sodium sulfate, or organic components such as calcium lactate, magnesium succinate, tartaric acid, sorbitol, mannitol, glucose or sucrose (Rathore and Nema, 2009).

Soft contact lenses as inserts. Soft contact lenses are composed of covalently crosslinked hydrophilic or hydrophobic polymers that form a three-dimensional network or matrix capable of retaining water, aqueous solution or solid components. A hydrophilic contact lens may be soaked in a drug solution, thereby absorbing the drug, but does not give precise delivery as compared to some other non-soluble ophthalmic inserts. The drug release from soft contact lenses is generally very rapid at the beginning, declining exponentially with time. The release rate can be decreased by incorporating the drug homogeneously during the manufacture or by adding a hydrophobic component (Rathore and Nema, 2009).

Soluble inserts. Soluble inserts are the oldest type of ophthalmic insert. They offer the great advantage of being entirely soluble so that they do not need to be removed from their site of application thus limiting the interventions to insertion only. They may contain natural polymers like collagen or synthetic or semi-synthetic polymers. Therapeutic agent is absorbed by soaking the insert in a solution containing the drug, drying and rehydrating before use on the eye. The amount of drug loaded depends on the amount of binding agent, concentration of the drug solution and soaking duration. Soluble synthetic polymers may include cellulose derivatives such as hydroxypropyl cellulose, methylcellulose, hydroxyethyl cellulose and hydroxypropyl cellulose, polyvinyl alcohol or ethylene vinyl acetate copolymer. Additives may include plasticizers such as polyethylene glycol, glycerin, propylene glycol, enteric coated polymers such as cellulose acetate phthalate, hydroxypropyl methylcellulose and phthalate, complexing agents such as polyvinyl pyrrolidone, and bioadhesives such as polyacrylic acids (Rathore and Nema, 2009).

Biodegradable ophthalmic inserts. Biodegradable or bioerodible inserts are composed of material homogeneous dispersion of a drug included or not into a hydrophobic coating which is substantially impermeable to the drug. They are made of the so-called biodegradable polymers. Successful biodegradable materials for ophthalmic use are the poly (orthoesters) and poly(orthocarbonates). The release of the drug from such a system is the consequence of the contact of the device with the tear fluid inducing a superficial diversion of the matrix (Rathore and Nema, 2009). Biodegradable inserts may contain cellulose derivatives, like hydroxypropyl methylcellulose (HPMC), hydroxyethyl cellulose (HEC), sodium carboxymethyl cellulose, ethyl cellulose, acrylates, like, polyacrylic acid and its cross-linked forms, Carbopol or Carbomer, chitosan, starch, for example, drum-dried waxy maize starch, and excipients, such as mannitol, sodium stearyl fumarate and magnesium stearate, polymers such as poly(alkyl cyanoacrylate), polylactic acid, poly(epsilon-caprolactone), poly(lactic-co-glycolic acid), chitosan, gelatin, sodium alginate and albumin (Barnaowski et al., 2014).

NACA (NPI-001) Oral dosage form. Oral dosage forms of NACA may include tablets, capsules, granules, solution, suspension and emulsion. One NACA dosage form, a 250 mg tablet, is described in US Patent 10590073. Similar dosage forms can be formulated for diNACA (NPI-002).

diNACA (NPI-002) as a prodrug to NACA, NAC. diNACA (NPI-002) may serve as a prodrug to NACA and NAC, as described in PCT/US21/14819.

In accordance with an embodiment, the present invention provides a method for the prevention, amelioration, or treatment of a disease or condition associated with oxidative stress in a subject comprising administration of a therapeutically effective amount of NACA, to increase the amount of glutathione expressed in the tissues of the subject.

As used herein, “active oxygen species” or “reactive oxygen species” are understood as transfer of one or two electrons produces superoxide, an anion with the form O2”, or peroxide anions, having the formula O2—” or compounds containing an O—O single bond, for example hydrogen peroxides and lipid peroxides. Such superoxides and peroxides are highly reactive and can cause damage to cellular components including proteins, nucleic acids, and lipids.

As used herein, the term “agent” refers to a therapeutically active compounds or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein, the terms “amelioration” or “treatment” is understood as meaning to lessen or decrease at least one sign, symptom, indication, or effect of a specific disease or condition. For example, amelioration or treatment of cystinosis can be to reduce, delay, or eliminate one or more signs or symptoms of cystinosis including, but not limited to, a reduction in vision, a reduction in overall visual acuity, a reduction in visual field, a reduction in deposits of cystine crystals in the cornea or other ocular tissue, a reduction in photophobia; or any other clinically acceptable indicators of disease state or progression. Amelioration and treatment can require the administration of more than one dose of an agent, either alone or in conduction with other therapeutic agents and interventions. Amelioration or treatment does not require that the disease or condition be cured.

As used herein, the term “Antioxidant” refers to a molecule for slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and N-acetylcysteine.

As used herein, the term “co-administration” refers to the administration of one or more agents to a subject such that the agents are present and active in the subject at the same time. Co-administration does not require a preparation of an admixture of the agents or simultaneous administration of the agents.

As used herein, the terms “effective amount” or “effective doses” refer to that amount of an agent to product the intended pharmacological, therapeutic or preventive results. The pharmacologically effective amount results in the amelioration of one or more signs or symptoms of a disease or condition or the advancement of a disease or conditions, or causes the regression of the disease or condition. For example, a therapeutically effective amount preferably refers to the amount of a therapeutic agent that decreases vision loss, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the phrase “oxidative stress related ocular disorders” includes, but is not limited to: cystinosis, cataract, macular degeneration including age-related macular degeneration, glaucoma, presbyopia, diabetic retinopathy, Lebers optic neuropathy, optic neuritis and retinitis pigmentosa.

As used herein, the terms “peroxidases” or “a peroxide metabolizing enzyme” refer to a large family of enzymes that typically catalyze a reaction of the form:

ROOR1+electron donor (2 e−)+2H+→ROH+R1OH

For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

As used herein, the term phrase “pharmaceutically acceptable carrier” refers to a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically are carriers acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperitoneal, intraocular, intravitreal, posterior juxtascleral, anterior juxtascleral, retrobulbar, suprachoroidal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, the term “plurality” is understood to mean more than one. For example, a plurality refers to at least two, three, four, five, or more.

As used herein, the term a “polypeptide” or “peptide” is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full-length proteins (e.g., fully processed proteins) as well as shorter amino acids sequences (e.g., fragments of naturally occurring proteins or synthetic polypeptide fragments).

As used herein, the term “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop cystinosis. The age of onset of one or more symptoms of the disease can sometimes be determined by the specific mutation. Prevention can include the delay of onset of one or more signs or symptoms of cystinosis and need not be prevention of appearance of at least one sign or symptom of the disease throughout the lifetime of the subject. Prevention can require the administration of more than one dose of an agent or therapeutic.

As used herein, the term “small molecule” refers to a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

As used herein, a subject “suffering from or suspected of suffering from” refers to a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition or syndrome. Methods for identification of subjects suffering from or suspected of suffering from conditions such as cystinosis are within the ability of those in the art. Subjects suffering from, and suspected of suffering from, a specific disease, condition, or syndrome are not necessarily two distinct groups.

As used herein, the term “superoxide dismutase” is understood as an enzyme that dismutation of superoxide into oxygen and hydrogen peroxide. Examples include, but are not limited to SOD1, SOD2, and SOD3. Sod1 and SOD3 are two isoforms of Cu-Zn-containing superoxide dismutase enzymes exists in mammals. Cu-Zn-SOD or SOD1, is found in the intracellular space, and extracellular SOD (ECSOD or SOD3) predominantly is found in the extracellular matrix of most tissues.

As used herein, the term “therapeutically effective amount,” refers to an amount of an agent which is effective, upon single or multiple does administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The present invention is directed to the use of NACA and/or diNACA to treat cystinosis. In one embodiment, the present invention includes a method for the treatment of cystinosis in a human that comprises administering to the human therapeutically effective amount of NACA and/or diNACA. In some embodiments, the NACA and/or diNACA is provided in or with a pharmaceutically acceptable carrier. In other embodiments, the NACA and/or diNACA is administered intraocularly, subretinally, intravitreally, posterior juxtasclerally, anterior juxtasclerally, retrobulbarly, suprachoroidally, orally, intravenously, intramuscularly, topically, ophthalmically, ocularly, sublingually, or rectally.

The thiol moiety of NAC, NACA, diNACA and their respective internal standards oxidizes quickly in plasma through formation of disulfides. In order to determine total NAC and total NACA levels in plasma, tris(2-carboxyethyl)phosphine (TCEP) is added during the extraction to reduce disulfide bonds (King et al., 2019). Ammonium bicarbonate is added to control sample pH near neutral, as TCEP will otherwise acidify the aliquoted samples and hinder derivatization. The free analyte is then derivatized to a stable thioether using 2-chloro-1-methylpyridinium iodide (CMPI). N-acetyl-L-cysteine is used as the reference standard. The assay would not discern the enantiomer, N-acetyl-D-cysteine, if present. A sample volume of 25.0 μL is aliquoted into a 1.2 mL 96-well plate to which is added, in sequence, 25.0 μL internal standard solution (1000 ng/mL NAC-D3 and 1000 ng/mL NACA-D3 in water), 50.0 μL of ammonium bicarbonate (100 mM), 5.0 μL CMPI (60 mM in water), and 5.0 μL of TCEP (60 mM in water). Samples are allowed to react for 30 minutes. To precipitate proteins, 500 μL of acetonitrile is then added to all samples. The plate is covered and the mixtures are shaken and centrifuged. A 50.0 μL aliquot of the supernatant is transferred from each well to a clean plate containing 400 μL of water-acetonitrile (25-75) in each well and mixed well prior to LC-MS injection.

Thioether derivatives of NAC and NACA for LCMS analyses

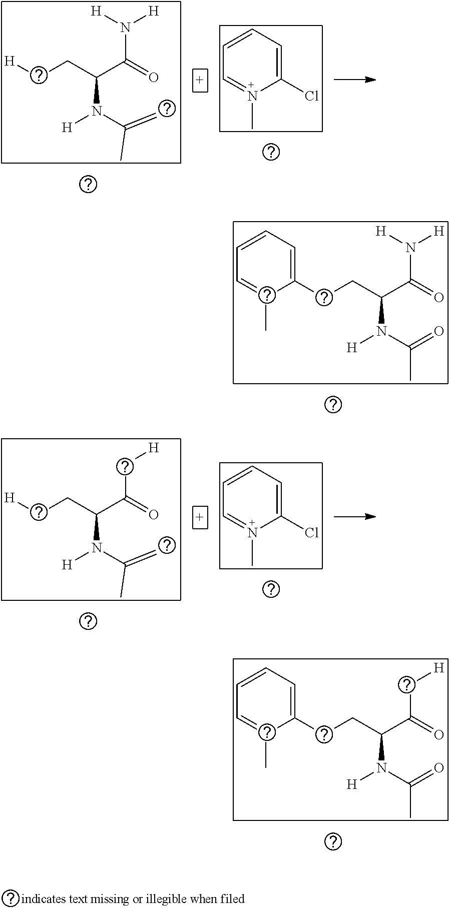

Samples were analyzed on a Waters Acquity liquid chromatograph interfaced with a Thermo Scientific TSQ Vantage triple quadrupole mass spectrometer with ESI ionization (King et al., 2019). Each extracted sample is injected (5.0 μL) onto a Waters BEH HILIC column (2.1×100 mm; 1.7 μm) equilibrated at 35° C. Mobile Phase A is ammonium formate (25 mM, pH 3.8). Mobile Phase B is acetonitrile. The LC gradient is tabled below:

| Time | Flow Rate | | |
|---|---|---|---|
| (mm) | (mL/min) | % MP A | % MP B |
| 0.00 | 0.500 | 25.0 | 75.0 |
| 2.30 | 0.500 | 25.0 | 75.0 |

The retention time, mass transition and precursor charge state for each compound are as follows. The masses below are for the CMPI thioether derivatives.

| | | | Product | |
|---|---|---|---|---|
| | Expected | Precursor | Observed | Charge |
| | Retention | Mass/ | Mass/ | State of |
| | Time | Charge | Charge | Precursor |
| Compound | (min) | (m/z) | (m/z) | Ion |
| N-Acetyl-L-Cysteine (NAC) | 1.90 | 255.080 | 126.16 | +1 |
| N-Acetylcysteine amide | 1.25 | 254.096 | 126.16 | +1 |
| (NACA) | | | | |
| N-Acetyl-L-Cys-D3 | 1.90 | 258.099 | 126.15 | +1 |
| N-Acetyl-L-Cysteine-D3 | 1.25 | 257.115 | 126.15 | +1 |

Peak area ratios from the calibration standard responses are regressed using a (1/concentration²) linear fit for N-Acetyl-L-Cysteine and N-Acetylcysteine amide. diNACA is quantitated in a similar manner.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the forgoing guidelines.

As used herein, the embodiments of this invention are defined to include pharmaceutically acceptable derivatives thereof. A “pharmaceutically acceptable derivative” means any pharmaceutically salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-napthalenesulfonate, nicotinate, nitrate, palmoate, phosphate, picrate, pivalate, propionate, salicylate, succinate, sulfate, tartrate, tosylate, and undeconaoate. Salts derived from appropriate bases include alkali metal (e.g., sodium), alkaline earth metal (e.g., magnesium), ammonium and N-(alkyl)4+ salts. This invention also envisions the quaternization of any basic nitrogen-containing groups of the compounds disclosed herein. Water or oil-soluble or dispersible products may be obtained by such quaternization.

The embodiments of the invention can, for example, be administered by injection, intraocularly, intravitreally, retrobulbarly, posterior juxtasclerally, anterior juxtasclerally, subretinally, topical ocularly, intravenously, intraarterially, subdermally, intramuscularly, or subcutaneously; or orally, buccally, nasally, transmucosally, directly to a diseased organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose.

Frequency of dosing will depend on the agent administered, the progression of the disease or condition in the subject, and other considerations known to those of skill in the art. For example, pharmacokinetic and pharmacodynamics considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night vision, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms and the judgment of the treating physician.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPAN® and/or other similar emulsifying agents or bioavailability enhancers which are commonly used in the manufacture of pharmaceutically acceptable solid, liquid, or other dosage forms may also be used for the purposes of formulation.

In one or more embodiments, NACA or diNACA is administered in daily doses of about 0.5 to 150 mg/Kg. In other embodiments, NACA or diNACA is administered two or three times daily. In another aspect, NACA or diNACA is administered with a second active agent selected from ascorbic acid, cysteamine (or any salt form), cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In some embodiments, the dose of NACA or diNACA for administration is, 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 102, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, the NACA or diNACA is administered prophylactically to prevent cystinosis.

In another embodiment, the present invention includes a method for the treatment of cystinosis consisting essentially of: identifying a human in need of treatment for cystinosis; and administering to the human a therapeutically effective amount of NACA or diNACA sufficient to treat cystinosis. It will be understood that, as with the other embodiments defined above, NACA or diNACA is administered in daily doses of about 0.5 to 150 mg/Kg. In another aspect, NACA or diNACA is administered two or three times daily. In another aspect, NACA is administered with a second active agent as disclosed above.

In another aspect, the dose of NACA or diNACA for administration is 100, 150, 150, 300, 333, 400, 500, 600, 700, 750, 800, 900, 1,000, 2,500, 5,000, 7,500, or 10,000 mg per dose. In another aspect, the dose for administration is 0.1-0.25, 0.1-0.4, 0.35-0.5, 0.5-1, 102, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, NACA or diNACA is administered prophylactically to prevent cystinosis.

In another aspect, NAC or cysteamine (or any salt form) is incorporated into a bioerodible or nonbioerodible ophthalmic, or ocular insert. The ophthalmic insert is placed on the surface of the eye under the eyelid. The Agent is eluted onto the surface of the eye over time and is absorbed into the eye, through the cornea, the major site of cystine deposits in the eye that can cause photophobia, which can be severe. The Agent disrupts the chemical structure of cystine, the result of which is the expulsion of cystine as a mixed dimer from the tissue, thereby ameliorating the ophthalmic effects of cystinosis.

As used herein, “susceptible to” or “prone to” or “predisposed to” a specific disease or condition or the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200% or more.

The NACA and diNACA formulations described herein may also be delivered to the eye via ocular topical, intravitreal injection, posterior juxtascleral injection, anterior juxtascleral injection, suprachoroidal, and periocular injection routes. In one embodiment of the present invention, the amount of active agent, or poorly water soluble agent, or biologic will be from about 0.001% to 30% weight to volume of the active agent in a solution for intravitreal administration. In other embodiments, the amount of active can be from 0.05% to 20% weight to volume and in some cases from 0.1% to 18% weight to volume. It is contemplated that any active agent that is poorly water soluble, or slightly soluble, may be included in the compositions of the present invention. In other instances, highly water-soluble active or inactive agents may also be included in the compositions of the present invention.

For example, the present invention that may be delivered is a thick or viscous vehicle that allows for extended release of the active agent. For example, the NACA or diNACA can be provided in any polyethyleneglycol (PEG) with a molecular weight greater than 500, 1000, 1,5000, or 2000 in the compositions and methods of the invention. PEGs for use in the compositions and methods of the invention can also include PEG 3000, PEG 4000, PEG 6000, PEG 8000, PEG 20000, or higher molecular PEGs may be utilized in the compositions and methods of the invention.

The NACA and/or diNACA formulations of the present invention provide a number of advantages over conventional formulations because PEGs can successfully solubilize poorly soluble compounds, allowing the preparation of an efficacious ophthalmologically acceptable intravitreal, posterior juxtascleral (PJ), anterior juxtascleral (AJ), and/or periocular formulation for local ocular delivery. Bioavailability of the drug can be modulated by controlling the molecular weight, or mixture of molecular weights, of the PEG used in the formulation. Furthermore, the preparation can be injected using a 27- or 30-gauge needle. Toxicity of the active agent can also be reduced or suitably modulated by extending its release. Another advantage of the present invention is that stability of biologics are improved in the PEG-based solid dosage form described herein.

The formulation of the invention may further comprise a lipid to modulate the delivery of the drug and to extend the duration. Some examples of a lipid include triglycerides, diglycerides, monoglycerides, propylene glycol esters, PEF esters of fatty acid and their mixtures. Preferred lipids include glyceryl monolaurate; glyceryl dilaurate; glyceryl monomyristate; glyceryl dimyristate; glyceryl monopalmitate; glyceryl dipalmitate; glyceryl monostearate; glyceryl distearate; glyceryl monooleate; glyceryl dioleate; glyceryl monolinoleate; glyceryl dilinoleate; glyceryl monoarachidate; glyceryl diarachidate; glyceryl monobehenate; glyceryl dibehenate; diethylene glycol monostearate; propylene glycol monostearate; glyceryl monostearate; glyceryl monolinoleate; glyceryl monooleate; glyceryl monopalmitate; and mixtures thereof. A preferred example of the lipid is glyceryl palmitostearate. The concentration of a lipid is generally less than 31 weight percent (wt %), but often less than 14 wt % and in some cases less than 8 wt %.

The specific dose level of the active agent for any particular human or animal depends upon a variety of factors, including the activity of the active compound used, the age, body weight, general health, time of administration, route of administration, and the severity of the pathologic condition undergoing therapy.

This present invention also relates to the general field of using NAC, NACA, diNACA, cysteamine (or cysteamine salts, e.g., the hydrochloride salt or other salts) or other cystine-depeting agents for treating ophthalmic, ocular or corneal effects of cystinosis by administration in an ophthalmic or ocular insert. Other cystine-depleting agents that may include but are not limited to those reported by Buchan et al., 2010; McCaughan et al., 2008; Omran et al., 2011a; Omran et al., 2011b; and Omran et al., 2011c, may be used in an ophthalmic or ocular insert.

### Example 1

### Cytotoxicity of NACA and diNACA in Cystinotic Fibroblast Cell Culture

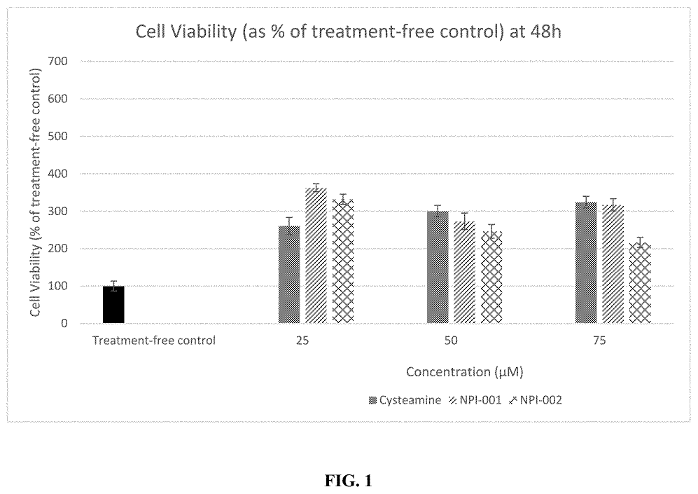

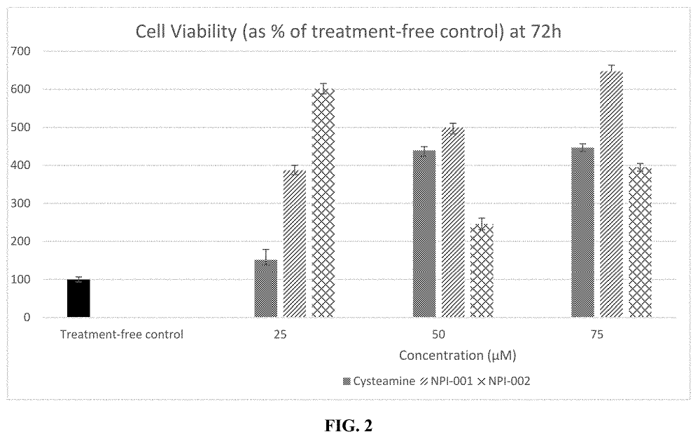

Appropriate concentrations of the NACA and diNACA were selected and incubated along with human cystinotic fibroblasts in media. Cystinotic fibroblasts (GM00008, Coriell Cell Repositories, NJ, USA) were cultured in 96 well plates incubated for 0-72 h in the presence of 25, 50 or 75 μM each of either cysteamine, NACA or diNACA. Media was then removed, and cells were incubated in 0.5mg/ml MTT (3-(4,5-dimethylthiazol-2-yl)-2,5-diphenyltetrazolium bromide) reconstituted in media. A colorimetric assay used reduction of the yellow tetrazolium salt (MTT) to a purple formazan salt in order to measure cellular metabolic activity as a proxy for cell viability. Treated cells were incubated in MTT for 4 hours at 37° C., after which time intracellular purple formazan salt crystals were visible under a microscope. MTT solution was then removed, and DMSO was added to each well in order to lyse cells and dissolve the salt crystals. After 1 hour incubation at 37° C., absorbance was measured on a multiwell plate reader (Biotek FL6000) at 570 nm, and percentage cell viability calculated. Exposure to all three concentrations of cysteamine, NACA (NPI-001) and diNACA (NPI-002) caused increased cell viability, i.e., no cytotoxicity (FIGS. 1 and 2). Surprisingly, NACA (NPI-001) at all concentrations was statistically superior to cysteamine at the same concentrations with regard to increasing human cystinotic cell viability after 72 hours (FIG. 2). Also surprisingly, diNACA (NPI-002) at the lowest concentration, 25 μM, was statistically superior to cysteamine at the same concentration, with regard to increasing human cystinotic cell viability after 48 or 72 hours (FIG. 1 and FIG. 2).

### Example 2

### Screening of Cystine-Depleting Activity of NACA and diNACA

A specialized in vitro human cystinotic cell-based model was used for determining cystine content (Omran et al., 2011c). Cystinotic fibroblasts (GM00008, Coriell Cell Repositories, NJ, USA) were seeded in a 25cm3 vented flask and allowed to reach approximately 80% confluence before the addition of the test articles; 50 μM of either cysteamine, NACA (NPI-001) and diNACA (NPI-002) in 4 cm3 Eagle's minimum essential media supplemented with 15% FBS, 200 U/ml penicillin, 200 μg/ml streptomycin and 2 mM glutamine. This was incubated at 37 ° C. and 5% CO2 for 24 h. The cells were harvested, frozen in liquid nitrogen and stored at −80° C. until the cysteine concentration was determined per quantity of protein.

The cells were recovered from storage at −80° C. and suspended in 100 μl mM N-ethyl malimide prepared in phosphate buffer (pH 7.6) followed by sonication for 10 seconds which was repeated 3 times with 20 second cooling intervals on ice. The solution was centrifuged at 800 G for 10 min at 4° C. (Biofuge primo R Heraeus centrifuge). To this cell supernatant (40 μl) was added 4 μl of 4M NaBH₄ in 7:3 0.1 M NaOH/DMSO. After 5 min incubation at room temperature, 800 μl of sodium acetate buffer (pH 4.7) was added. A 5 μl volume of the diluted solution was added to 100 μl of 0.6mg/ml solution of Papain-SSCH₃ in a 96 well plate and incubated for one hour at room temperature. A 100 μl volume of 4.9 mM L-benzoylarginine p-nitroanilide (L-BAPNA) solution in sodium acetate buffer (pH 4.0) was added to each well of the 96 well plate, gently mixed and incubated for further one hour at room temperature. Absorption at 410 nm was measured and the cysteine levels were calculated by comparison to known cysteine standard.

The protein concentration in every sample was measured according to Bradford method. Briefly, 200 μl of Bradford reagent was added to 5 μl of cell supernatant in each well of a 96 well plate and incubated for 5 minutes at room temperature and the absorption at 595 nm was measured. The protein concentration was calculated using a range of concentrations of bovine serum albumin as a standard. The cysteine levels were determined following normalization to μM cysteine per mg of protein.

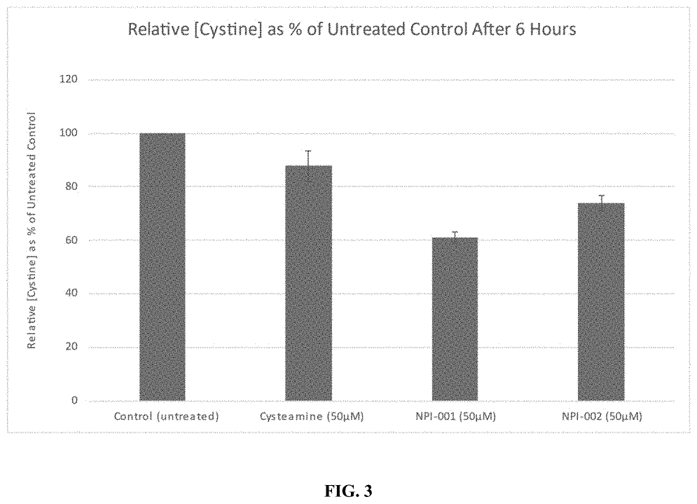

FIG. 3 shows relative cystine concentration as percent of control in human cystinotic fibroblast cell culture after exposure to 50 μM cysteamine, NACA (NPI-001) or diNACA (NPI-002) for 6 hours. Surprisingly, both NACA (NPI-001) and diNACA (NPI-002) were statistically superior to cysteamine after 6 hours.

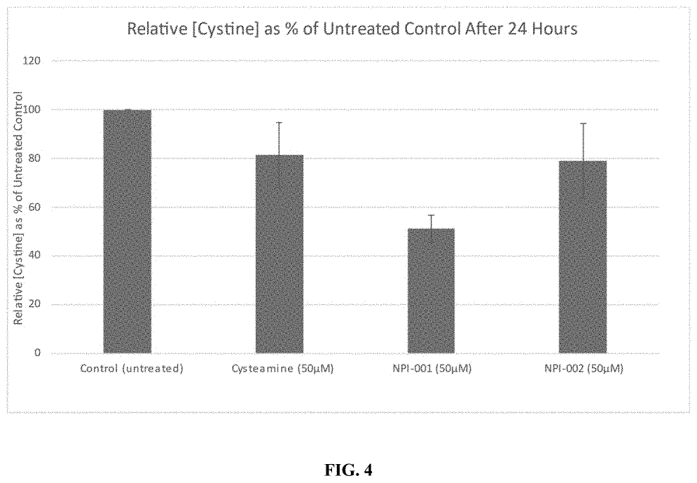

FIG. 4 shows relative cystine concentration as percent of control in human cystinotic fibroblast cell culture after exposure to 50 μM cysteamine, NACA (NPI-001) or diNACA (NPI-002) for 24 hours. Surprisingly, NACA (NPI-001) was statistically superior and diNACA was superior to cysteamine in increasing human cystinotic cell viability after 24 hours.

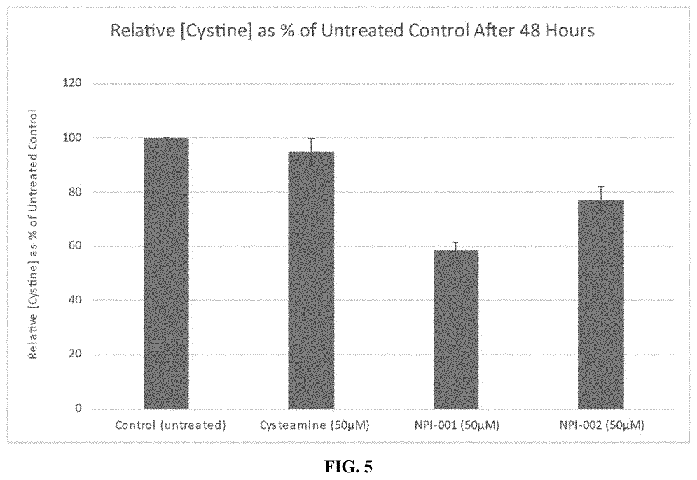

FIG. 5 shows relative cystine concentration as percent of control in human cystinotic fibroblast cell culture after exposure to 50 μM cysteamine, NACA (NPI-001) or diNACA (NPI-002) for 48 hours. Surprisingly, both NACA (NPI-001) and diNACA (NPI-002) were statistically superior to cysteamine and after 48 hours.

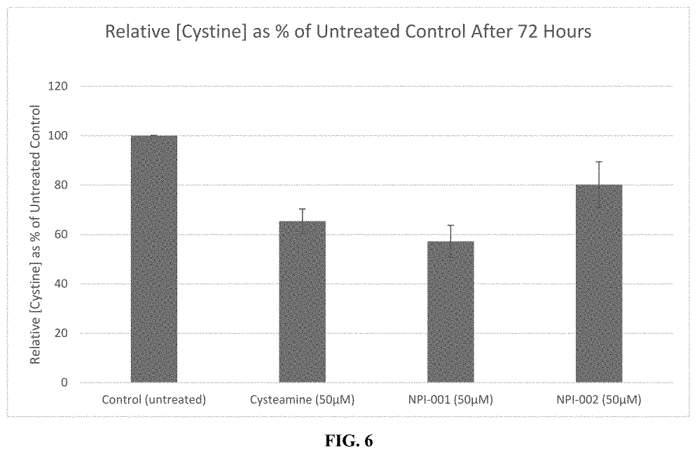

FIG. 6 shows relative cystine concentration as percent of control in human cystinotic fibroblast cell culture after exposure to 50 μM cysteamine, NACA (NPI-001) or diNACA (NPI-002) for 72 hours. Surprisingly, NACA (NPI-001) was superior to cysteamine and after 72 hours and diNACA (NPI-002) exhibited depletion of 20% cystine compared to control.

### Example 3

### Ophthalmic Tolerability of 1% NPI-001, 1% diNACA, 1% NAC or vehicle

To simulate topical ocular exposure to an ophthalmic insert containing AGENT, prototype ophthalmic formulations were prepared containing either 1% NPI-001, 1% diNACA, 1% NAC or vehicle and tested to evaluate ocular tolerability. Three rabbits per group were administered 1% NPI-001, 1% di-NAC, 1% NAC in a prototype vehicle, via bilateral topical administration four times a day for 6 days, and once on Day 7. Mild discharge was observed at low frequencies in all four administration groups, and mild hyperemia was observed at low frequencies in the 1% NAC and vehicle administration groups. No indications of chemosis were seen in any of the administration groups. Because the vehicle treatment showed similar or greater frequency of mild ocular irritation indicated by discharge or hyperemia, the ocular irritation seen in the test agent administration groups are likely not attributable to the test agent themselves, but most likely due to the vehicle (Tables 1-3).

TABLE 1
Daily Draize Scores for Discharge

| | Discharge | | | | | | | | | |
|---|---|---|---|---|---|---|---|---|---|---|
| | | | Day | | | | | | | |
| | Animal | Eye | B | 1 | 2 | 3 | 4 | 5 | 6 | 7 |
| 1% NPI-001 | 1337 | L | 0 | 0 | 0 | 0 | 0 | 0 | 0 | 0.5 |
| | | R | 0 | 0 | 0 | 0 | 0 | 0 | 0 | 0 |
| | 1338 | L | 0 | 0 | 0 | 0 | 0 | 0 | 0 | 0 |
| | | R | 0 | 0 | 0 | 0 | 0 | 0 | 0.5 | 0 |
| | 1339 | L | 0 | 0 | 0 | 0 | 0 | 0 | 0 | 0.5 |
| | | R | 0 | 0 | 0 | 0 | 0 | 0 | 0 | 0 |
| 1% diNACA | 1340 | L | 0 | 0 | 0 | 0 | 0 | 0 | 0 | 0 |
| | | R | 0 | 0 | 0 | 0 | 0 | 0 | 0 | 0 |
| | 1341 | L | 0 | 0 | 0 | 0 | 1 | 0 | 0 | 0 |
| | | R | 0 | 0 | 0 | 0 | 0 | 0 | 0 | 0 |
| | 1342 | L | 0 | 0 | 0 | 0 | 1 | 0 | 0.5 | 0 |
| | | R | 0 | 0 | 0 | 0 | 0.5 | 0 | 0.5 | 0.5 |
| 1% NAC | 1343 | L | 0 | 0 | 0 | 0 | 0 | 0.5 | 0 | 0.5 |
| | | R | 0 | 0 | 0 | 0 | 1 | 0 | 0 | 0.5 |
| | 1344 | L | 0 | 0 | 0.5 | 0 | 0 | 0 | 1 | 1 |
| | | R | 0 | 0 | 0.5 | 0 | 0 | 0 | 1 | 0.5 |
| | 1345 | L | 0 | 0 | 0 | 0 | 0 | 0 | 0 | 0 |
| | | R | 0 | 0 | 0 | 0 | 0.5 | 0 | 1 | 0.5 |
| Vehicle | 1346 | L | 0 | 0 | 0.5 | 0 | 0 | 0 | 0 | 0.5 |
| | | R | 0 | 0 | 0 | 0 | 0 | 0 | 0 | 1 |
| | 1347 | L | 0 | 0 | 0 | 0 | 0 | 0 | 0 | 0 |
| | | R | 0 | 0 | 0 | 0 | 0 | 0 | 0 | 0 |
| | 1348 | L | 0 | 0 | 0.5 | 0 | 0 | 0 | 0.5 | 0.5 |
| | | R | 0 | 0 | 0.5 | 0 | 0 | 0 | 0 | 0.5 |

TABLE 2
Daily Draize Scores for Chemosis

| | Chemosis | | | | | | | | | |
|---|---|---|---|---|---|---|---|---|---|---|
| | | | Day | | | | | | | |
| | Animal | Eye | B | 1 | 2 | 3 | 4 | 5 | 6 | 7 |
| 1% NPI-001 | 1337 | L | 0 | 0 | 0 | 0 | 0 | 0 | 0 | 0 |
| | | R | 0 | 0 | 0 | 0 | 0 | 0 | 0 | 0 |
| | 1338 | L | 0 | 0 | 0 | 0 | 0 | 0 | 0 | 0 |
| | | R | 0 | 0 | 0 | 0 | 0 | 0 | 0 | 0 |
| | 1339 | L | 0 | 0 | 0 | 0 | 0 | 0 | 0 | 0 |
| | | R | 0 | 0 | 0 | 0 | 0 | 0 | 0 | 0 |
| 1% diNACA | 1340 | L | 0 | 0 | 0 | 0 | 0 | 0 | 0 | 0 |
| | | R | 0 | 0 | 0 | 0 | 0 | 0 | 0 | 0 |
| | 1341 | L | 0 | 0 | 0 | 0 | 0 | 0 | 0 | 0 |
| | | R | 0 | 0 | 0 | 0 | 0 | 0 | 0 | 0 |
| | 1342 | L | 0 | 0 | 0 | 0 | 0 | 0 | 0 | 0 |
| | | R | 0 | 0 | 0 | 0 | 0 | 0 | 0 | 0 |
| 1% NAC | 1343 | L | 0 | 0 | 0 | 0 | 0 | 0 | 0 | 0 |
| | | R | 0 | 0 | 0 | 0 | 0 | 0 | 0 | 0 |
| | 1344 | L | 0 | 0 | 0 | 0 | 0 | 0 | 0 | 0 |
| | | R | 0 | 0 | 0 | 0 | 0 | 0 | 0 | 0 |
| | 1345 | L | 0 | 0 | 0 | 0 | 0 | 0 | 0 | 0 |
| | | R | 0 | 0 | 0 | 0 | 0 | 0 | 0 | 0 |
| Vehicle | 1346 | L | 0 | 0 | 0 | 0 | 0 | 0 | 0 | 0 |
| | | R | 0 | 0 | 0 | 0 | 0 | 0 | 0 | 0 |
| | 1347 | L | 0 | 0 | 0 | 0 | 0 | 0 | 0 | 0 |
| | | R | 0 | 0 | 0 | 0 | 0 | 0 | 0 | 0 |
| | 1348 | L | 0 | 0 | 0 | 0 | 0 | 0 | 0 | 0 |
| | | R | 0 | 0 | 0 | 0 | 0 | 0 | 0 | 0 |

TABLE 3
Daily Draize Scores for Hyperemia

| | Hyperemia | | | | | | | | | |
|---|---|---|---|---|---|---|---|---|---|---|
| | | | Day | | | | | | | |
| | Animal | Eye | B | 1 | 2 | 3 | 4 | 5 | 6 | 7 |
| 1% NPI-001 | 1337 | L | 0 | 0 | 0 | 0 | 0 | 0 | 0 | 0 |
| | | R | 0 | 0 | 0 | 0 | 0 | 0 | 0 | 0 |
| | 1338 | L | 0 | 0 | 0 | 0 | 0 | 0 | 0 | 0 |
| | | R | 0 | 0 | 0 | 0 | 0 | 0 | 0 | 0 |
| | 1339 | L | 0 | 0 | 0 | 0 | 0 | 0 | 0 | 0 |
| | | R | 0 | 0 | 0 | 0 | 0 | 0 | 0 | 0 |
| 1% diNACA | 1340 | L | 0 | 0 | 0 | 0 | 0 | 0 | 0 | 0 |
| | | R | 0 | 0 | 0 | 0 | 0 | 0 | 0 | 0 |
| | 1341 | L | 0 | 0 | 0 | 0 | 0 | 0 | 0 | 0 |
| | | R | 0 | 0 | 0 | 0 | 0 | 0 | 0 | 0 |
| | 1342 | L | 0 | 0 | 0 | 0 | 0 | 0 | 0 | 0 |
| | | R | 0 | 0 | 0 | 0 | 0 | 0 | 0 | 0 |
| 1% NAC | 1343 | L | 0 | 0 | 0 | 0 | 0 | 0 | 0 | 1 |
| | | R | 0 | 0 | 0 | 0 | 0 | 0 | 0 | 0.5 |
| | 1344 | L | 0 | 0 | 0 | 0.5 | 0 | 0 | 0 | 1 |
| | | R | 0 | 0 | 0.5 | 0.5 | 0 | 0 | 0 | 1 |
| | 1345 | L | 0 | 0 | 0 | 0.5 | 0 | 0 | 0 | 0.5 |
| | | R | 0 | 0 | 0.5 | 0 | 0 | 0 | 0 | 0.5 |
| Vehicle | 1346 | L | 0 | 0 | 0 | 0 | 0 | 0 | 0 | 0.5 |
| | | R | 0 | 0 | 0 | 0 | 0 | 0 | 0 | 0.5 |
| | 1347 | L | 0 | 0 | 0 | 0 | 0 | 0 | 0 | 0 |
| | | R | 0 | 0 | 0 | 0 | 0 | 0 | 0 | 0 |
| | 1348 | L | 0 | 0 | 0 | 0 | 0 | 0 | 0 | 0 |
| | | R | 0 | 0 | 0 | 0 | 0 | 0 | 0 | 0.5 |

NACA was detectable in aqueous humor of rabbit eyes administered either 1% NACA or 1% diNACA.

### Example 4

### NACA (NPI-001), diNACA (NPI-002), NAC or cysteamine ophthalmic insert.

Ophthalmic insert will be prepared that is either a biodegradable insert or nonbiodegradable insert.

Nonbioerodible insert will be composed of contact lens polymer matrix, kidney- or wafer-shaped, similar in dimensions to half of a contact lens, loaded with Agent, i.e., either NACA (NPI-001), diNACA (NPI-002), NAC or cysteamine (or any salt form). It is produced as a single entity, i.e., a solid, non-erodible matrix. The nonbioerodible matrix will be similar in chemistry to non-hydrogel contact lens materials, with the Agent being contained in the liquid formulation prior to the molding into a solid.

Bioerodible insert will be composed of bioerodible or biodegradable resin or polymer matrix, kidney- or wafer-shaped, similar in dimensions to half of a contact lens, loaded with Agent, i.e., either NACA (NPI-001), diNACA (NPI-002), NAC or cysteamine (or any salt form).

Selection of bioerodible or nonbioerodible insert to carry forward in nonclinical and clinical studies will be based on pharmaceutical properties such as in vitro release, stability and manufacturability.

Bioerodible or nonbioerobile insert will be applied non-invasively to the superior sclera under the eyelid and elutes drug over time via topical administration to the eye. The matrix is molded to a curved shape to fit over the scleral surface of the eyeball, with smooth surfaces and edges, and additional design features that interact with the eyelid to maintain its position under the eyelid during wear. The Agent elutes over time, providing sustained delivery to the ocular surface and eventual penetration of Agent into ocular surfaces including cornea, the major site of cystine crystals. The Insert offers a patient a more convenient sustained delivery of drug compared to frequent eyedrop applications (Rathore and Nema, 2009).

### Example 5

### In vitro Release of NACA (NPI-001), diNACA (NPI-002), NAC or Cysteamine from Ophthalmic Insert

AGENT: NACA, diNACA, NAC and/or cysteamine (or any salt form). The Agent will be loaded into the insert. The insert will be placed in pH 7 buffer (phosphate), stirred with agitation, and samples taken over time, e.g., 15 minutes, 0.5, 1, 2, 3, 4, 8, 12, 24, 48, 72 hours. Buffer will be replenished with the same volume as sample taken. Samples will be analyzed for NACA, diNACA, NAC, and/or cysteamine based on methodology developed by King et al., 2019, or for cysteamine using similar methodology. The in vitro release rate will be determined. Release rate may be adjusted by modification of formulation components. Tentative acceptance criteria for drug product will be developed (Table 4).

TABLE 4
Example Acceptance Criteria for Ophthalmic Insert.

| Test | Method | Acceptance Criteria |
|---|---|---|
| Appearance of Insert | Visual Assessment | Conforms |
| AGENT Identification A | HPLC | Retention time conforms to |
| | | reference standard |
| AGENT Identification B | HPLC Photodiode | UV spectrum conforms to |
| | Array | reference standard |
| AGENT Related substances | HPLC | Report percent impurities |
| | | Report Total impurities percent |
| AGENT Assay* | HPLC | 90%-110% of label claim* |
| In Vitro Release Rate of Insert (only for | HPLC | Meets Requirements |
| stability testing) | | |
| Content Uniformity of AGENT | HPLC as per USP | Meets Requirements |
| | <905> | |
| Dimensions of Insert | Report | Report |
| Weight range of Insert | Report | Report |
| Sterility | USP <51> | Meets requirements |

*Concentration to be determined based on toxicology study results; AGENT: NAC, NACA, diNACA, NAC or cysteamine (or any salt form).

### Example 6

### Stability of Ophthalmic Insert

AGENT: NACA, diNACA, NAC, cysteamine (or any salt form) or any cystine-depleting agent. Insert containing AGENT will be stored at conditions compliant with International Committee on Harmonization ICH), e.g., at 25±2° C./60±5% RH for 2 years and 40±2° C./75±5% RH for 6 months and tested at time=0, 3, 6, 9, 12, 18 and 24 months. If specifications are not met over time, the inserts will then be stored in sealed hermetic pouches to prevent moisture loss and retested.

### Example 7

### In Vivo Tolerability and Release of NACA (NPI-001), DiNACA (NPI-002), NAC or Cysteamine from Ophthalmic Insert

AGENT: NACA, diNACA, NAC, cysteamine (or any salt form) or any cystine-depleting agent. Agent will be incorporated into rabbit inserts, identical in composition to human ophthalmic inserts, but conformed to fit onto the rabbit corneal surface. Under sedation, the rabbit-custom insert (custom size prepared for rabbit eye) will be placed on the surface of the rabbit eye, under the nictitating membrane. In order to prevent the insert from coming out of the eye, a partial tarsorrhaphy will performed by placing 2 small sutures in the outer corner of the eyelid, allowing the eye to partially open and blink normally. A small amount of bacitracin ophthalmic ointment will be administered to the suture site on the external lid. Post-insertion, the animals will be observed at least twice daily, for any signs of pain, infection, or irritation. Rabbits will be assessed, allowing for minimal lid swelling and ocular discharge due to procedure, for hyperemia, chemosis, discharge, corneal opacity, aqueous cells/flare and light reflex. The animals most affected will be chosen for the 24-hour removal/euthanization timepoint. Based on prototype experiments, the rabbit ring should be fairly well-tolerated after 24 or 48 hours of wear, with the exception of mild conjunctival congestion in all animals which is likely associated with a response to the physical presence of the rabbit ring. Quantitation of AGENT by validated HPLC/MS/MS (King et al., 2019) will be conducted for tissues including: tears, aqueous humor (AH), cornea, vitreous humor, and retina after 24 or 48 hours of wear. AGENT delivery to AH and cornea will be assessed. The presence of AGENT will be assessed in all ocular tissues using LCMS (e.g., King et al., 2019). No AGENT will be detected in ocular tissue of control animals. When placed under the eyelid of rabbits, AGENT will likely be delivered in high concentration to the AH and cornea as well as other ocular tissues.

### Example 8

### Nonclinical Study of NACA, diNACA or NAC

AGENT: NACA, diNACA, NAC, cysteamine (or any salt form) or any cystine-depleting agent.

Study Title: Six-month topical ocular repeat-dose study in rabbits with a three-month interim and a one month recovery.

Objective: To evaluate safety of daily (QID) topical administration of AGENT (formulations containing 0, 1, 5 and 10% AGENT in 0.05% phosphate buffer) drops in rabbits over six months with a three-month interim and a 1 month recovery at the high dose. An additional objective will be evaluation of toxicokinetics.

Regulatory Compliance: This GLP study will be conducted in compliance with testing facility SOPs and all animals will be treated in accordance with the ARVO Statement for the Use of Animals in Ophthalmic and Vision Research. The study will be conducted in accordance with the FDA's Good Laboratory Practice for Nonclinical Laboratory Studies, Code of Federal Regulations, Title 21 Part 58, and any applicable amendments, or equivalent international standard.

Testing Facility: GLP CRO. Test System. Species: Dutch-Belted Rabbits. Number of Animals: 80; 90 screened. Sex & Age: Adult males and females, evenly divided among treatment groups

Study Design. Rabbits with normal veterinary, ophthalmic exams and screening clinical pathology values will be recruited to the study (Table 5). For baseline screening and all subsequent procedures, anesthesia will be achieved and pupil dilation with appropriate medicaments. Rabbits will be rank ordered by weight and sex and then randomized by a block randomization procedure.

Test Article Delivery: Test article will be applied as eye drops to both eyes four times daily (Table 5).

Clinical observations: General wellbeing will be assessed twice daily by cage side observation beginning one week prior to dosing and extending to study terminus.

Food consumption: Food consumption will be monitored weekly.

Body Weights: Body weights will be collected at ophthalmic exam intervals (Table 6).

Tonometry: Intraocular pressure (TOP) will be measured OU using a TonoVet tonometer set to the dog (d) calibration setting. Three measures will be taken from each eye at each ophthalmic examination time point (Table 6) and the mean IOP defined.

Ophthalmic examinations: At designated time points (Table 6) slit lamp biomicroscopy and retinoscopy will be performed in both eyes (OU) and findings graded using a modified Hackett-McDonald scoring system and composite clinical scores will be determined. At baseline, 3 and 6 months, will evaluate ERGs in control and high dose group.

TABLE 5
Treatment Assignment

| | | | DRUG | | | | | |
|---|---|---|---|---|---|---|---|---|
| Group | Sacrifice | N | Treatment | Route | Volume | Frequency | Dosing | Terminus |
| 1 | 6 mos | 3M/3F | 0% | Topical | 50 μL | QID | Day 1- | Day 180 |
| | | | | Ocular | | | 180 | |
| | 3 mos | 3M/3F | | Topical | 50 μL | QID | Day 1- | Day 90 |
| | | | | Ocular | | | 90 | |
| | 7 mos | 2M/2F | | Topical | 50 μL | QID | Day 1- | Day 210 |
| | | | | Ocular | | | 180 | |
| 2 | 6 mos | 3M/3F | 0.1% | Topical | 50 μL | QID | Day 1- | Day 180 |
| | | | | Ocular | | | 180 | |
| | 3 mos | 3M/3F | | Topical | 50 μL | QID | Day 1- | Day 90 |
| | | | | Ocular | | | 90 | |
| 3 | 6 mos | 3M/3F | 1% | Topical | 50 μL | QID | Day 1- | Day 180 |
| | | | | Ocular | | | 180 | |
| | 3 mos | 3M/3F | | Topical | 50 μL | QID | Day 1- | Day 90 |
| | | | | Ocular | | | 90 | |
| 4 | 6 mos | 3M/3F | 5% | Topical | 50 μL | QID | Day 1- | Day 180 |
| | | | | Ocular | | | 180 | |
| | 3 mos | 3M/3F | | Topical | 50 μL | QID | Day 1- | Day 90 |
| | | | | Ocular | | | 90 | |
| 5 | 6 mos | 3M/3F | 10% | Topical | 50 μL | QID | Day 1- | Day 180 |
| | | | | Ocular | | | 180 | |
| | 3 mos | 3M/3F | | Topical | 50 μL | QID | Day 1- | Day 90 |
| | | | | Ocular | | | 90 | |
| | 7 mos | 2M/2F | | Topical | 50 μL | QID | Day 1- | Day 210 |
| | | | | Ocular | | | 180 | |

Clinical Pathology: Samples for analysis of hematology, clinical chemistry, coagulation, and urinalysis parameters will be evaluated at designated time points (Table 5).

Toxicokinetics: A blood sample (2 mL) will be collected at designated time points (Table 6) for serum preparation Serum aliquots will be transferred to pre-labeled cryotubes and stored and shipped below −70° C. to Sponsor designated laboratory for determination of serum test article concentrations by validated analytical procedure and standard toxicokinetic parameters for NPI-001 and NAC calculated Incurred sample reanalysis (ISR) will be performed.

TABLE 6
Schedule

| | Study Day | | | | | | | | |
|---|---|---|---|---|---|---|---|---|---|
| | Pre | 0 | 28 | 56 | 84 | 112 | 140 | 168 | 196 |
| | Study Month | | | | | | | | |
| Event | Pre | 0 | 1 | 2 | 3 | 4 | 5 | 6 | 7 |
| Dosing-topical ocular | X | X | X | X | X | X | X | X | — |
| Daily observations | X | X | X | X | X | X | X | X | X |
| Detailed clinical obs* | X | X | X | X | X | X | X | X | X |
| Food consumption | — | X | X | X | X | X | X | X | X |
| Body weight* | — | — | — | — | — | — | — | X | X |
| Tonometry | X | — | — | — | — | — | — | X | X |
| Slit lamp | X | — | X | X | X | X | X | X | X |
| ERG | X | | | | X | | | X | |
| Clinical pathology** | X | — | — | — | — | — | — | X | X |
| Toxicokinetics*** | X | — | — | — | X | | — | X | X |
| Necropsy** | — | — | — | — | X**** | — | — | X | X***** |
| Organ weights | — | — | — | — | X**** | — | — | X | X***** |
| Enucleation | — | — | — | — | X**** | — | — | X | X***** |

*Detailed clinical observations and body weights will be performed weekly
**Baseline clinical pathology will be evaluated in screened rabbits; clinical pathology and histology to be performed on day 84 (3 mo), day 168 (main) and day 196 (recovery)
***Toxicokinetic time points collected at predose, 0.25, 0.5, 1, 2, 4 and 8 hours postdose
****For 3-month interim
*****For 1-month recovery

Eye Collection: At designated time points (Table 6) rabbits will be euthanized with appropriate medicament while under anesthesia and eyes immediately enucleated. For the right eyes (OD), excess orbital tissue will be trimmed and placed in Davidson's fixative at room temperature for 48 hours then transferred and stored at 70% ethanol prior to shipment to GLP Laboratory for embedding in paraffin, sectioning (3 stepped sections), staining with Hematoxylin and Eosin (H&E), and analysis by a board-certified veterinary pathologist. Aqueous Humor, Vitreous humor, Lens and Retina/Choroid will be collected from left eyes (OS) using appropriate methods. Samples will be stored and shipped frozen for bioanalytical analysis for AGENT (LLOQ will be provided).

Necropsy: Complete necropsies will be performed on animals euthanized at scheduled time points (Table 3) or dying spontaneously. The skin will be reflected from a ventral midline incision and subcutaneous masses identified and correlated with antemortem findings. The abdominal, thoracic, and cranial cavities will be examined for abnormalities, and organs removed, examined, and tissues collected as specified in Table 7.

Histopathology: After enculeations are completed, a necropsy will be performed. Histopathology will be assessed only on ocular tissues and gross lesions. All other tissues will be preserved, but histopathology conducted only if warranted. Tissues for histology will be harvested and fixed in 10% neutral buffered formalin (NBF) for shipment to designated histology lab for hematoxylin and eosin (H&E) and immunohistochemistry (IHC) processing and analysis by a board-certified veterinary pathologist. Three stepped sections will be taken through each specimen and mounted for each stain.

TABLE 7
Necropsy Specimen Collection and Analysis.

| | | | | | Immuno- |
|---|---|---|---|---|---|
| | | Organ | Collected for | Microscopic | Histochemistry |
| Sample | Tissue | Weight | Post-fixation | Examination | * |
| 1 | Right eye | X | X | X | |
| 2 | Left eye | X | X | X | |
| 3 | Right eyelids | — | X | X | |
| 4 | Left eyelids | — | X | X | |
| 5 | Lung | X | X | X | |
| 6 | Heart | X | X | ** | |
| 7 | Aorta | — | X | ** | |
| 8 | Liver | X | X | ** | |
| 9 | Gall bladder | — | X | ** | |
| 10 | Pancreas | — | X | ** | |
| 11 | Submandibular salivary glands | — | X | ** | |
| 12 | Parotid salivary glands | — | X | ** | |
| 13 | Tongue | — | X | ** | |
| 14 | Esophagus | — | X | ** | |
| 15 | Stomach | — | X | ** | |
| 16 | Small intestine | — | X | ** | |
| 17 | Large intestine | — | X | ** | |
| 18 | Kidney | X | X | ** | |
| 19 | Urinary bladder | — | X | ** | |
| 20 | Testis | — | X | ** | |
| 21 | Ovary | — | X | ** | |
| 22 | Thyroid | — | X | ** | |
| 23 | Adrenal gland | — | X | ** | |
| 24 | Parathyroid gland | — | X | ** | |
| 25 | Pituitary | — | X | ** | |
| 26 | Mesenteric lymph node | — | X | ** | |
| 27 | Submandibular lymph node | — | X | ** | |
| 28 | Thymus | — | X | ** | |
| 29 | Spleen | X | X | ** | |
| 30 | Skin | — | X | ** | |
| 31 | Brain | X | X | ** | |
| 32 | Spinal cord | — | X | ** | |
| 33 | Sciatic nerve | — | X | ** | |
| 34 | Skeletal muscle | — | X | ** | |
| 35 | Bone marrow | — | X | ** | |
| 36 | All gross lesions | — | X | ** | |

* Immunohistochemistry (e.g. Ki67 for proliferation) to be conducted, guided by H&E findings;
** Histopathology will be assessed only on ocular tissues and gross lesions. All other tissues will be preserved, but histopathology conducted only if warranted.

Test Article Collection and Analysis: One vial/group (unopened) will also be retained and sent for concentration and homogeneity analysis on dosing day 0 and 84.

Study Report: A report detailing methods, in-life exam findings and specimen collection will be submitted to the Sponsor as draft audited report following in-life study completion, followed by finalized report inclusive of subcontracted lab reports and Sponsor input. The 3-month audited draft report will be amended to the Sponsor's IND by the Sponsor. SEND datasets will be generated and submitted along with the 3-month interim and 6-months final report, which will ultimately be amended to the Sponsor's IND by the Sponsor.

### Example 9

### Phase 2/3 3-Month Clinical Study with AGENT Insert (AGENT: NACA, diNACA, NAC or Cysteamine (or any Salt Form)

Clinical evaluation of an ophthalmic insert may follow example Phase 2/3 as per clinical Study Synopsis below (

AGENT: NACA, diNACA, NAC, cysteamine (or any salt form) or any cystine-depleting agent.).

Name of Sponsor/Company: Nacuity Pharmaceuticals Inc.
Name of Investigational Product: AGENT Ophthalmic Insert
Name of Active Ingredient: AGENT
Title of Study: A Phase 2/3 Study of the Safety and Efficacy of AGENT Ophthalmic Insert
compared to Placebo or active comparator Insert for Treatment of Ocular Effects of
Cystinosis
Study Number: C-XX-XX

| | |
|---|---|
| Study Period: 3 months | Phase of Development: Phase 2/3 |

Primary Objectives:
Evaluate the safety of AGENT Ophthalmic Insert in cystinosis subjects compared to
Placebo Insert based on adverse events, BCVA, slit lamp biomicroscopy and dilated
fundus exam.
Evaluate efficacy of AGENT Ophthalmic Insert based on reduction of In-Vivo Confocal
Microscopy total score (IVCM Score*) compared to Placebo Insert.
Exploratory Objectives:
Evaluate efficacy of AGENT Ophthalmic Insert based on comparison of the mean density
of inflammatory cells (IVCM-inf)** at each visit versus IVCM-inf at baseline.
Evaluate efficacy of AGENT Ophthalmic Insert based on comparison of IVCM-inf of
subjects treated with AGENT Ophthalmic Insert versus subjects treated with Placebo Insert
at each visit.
Evaluate efficacy of AGENT Ophthalmic Insert based on comparison of the anterior
segment optical coherence tomography (AS-OCT**) at each visit versus AS-OCT at
baseline.
Evaluate efficacy of AGENT Ophthalmic Insert based on comparison of AS-OCT of
subjects treated with AGENT Ophthalmic Insert versus subjects treated with Placebo Insert
at each visit.
Evaluate efficacy of AGENT Ophthalmic Insert based on comparison of the clinician-
assessed photophobia** at each visit versus baseline.
Evaluate efficacy of AGENT Ophthalmic Insert based on comparison of the clinician-
assessed photophobia of subjects treated with AGENT Ophthalmic Insert versus subjects
treated with Placebo Insert at each visit.
Evaluate efficacy of AGENT Ophthalmic Insert based on comparison of the subject-
assessed photophobia** at each visit versus baseline.
Evaluate efficacy of AGENT Ophthalmic Insert based on comparison of the subject-
assessed photophobia of subjects treated with AGENT Ophthalmic Insert versus subjects
treated with Placebo Insert at each visit.
Evaluate the treatment acceptability via questionnaire for subjects treated with AGENT
Ophthalmic Insert versus Placebo Insert.
(*Labbe et al., 2009; **Liang et al., 2015)
Methodology:
This study will examine the safety and efficacy of AGENT Ophthalmic Insert treatment for
ocular cystinosis in pediatric and adult subjects versus Placebo Insert.
Efficacy will be assessed based on IVCM Score, AS-OCT, and self- and investigator-assessed
photophobia. Subjects must have a baseline IVCM Score ≥4. A decrease in IVCM total score
from baseline indicates a reduction in corneal crystals.
Subjects will be randomized to either AGENT Ophthalmic Insert or Placebo Insert (2:1) for 3
months.
Safety will be assessed based on evaluation of adverse events, BCVA, slit lamp biomicroscopy
and dilated fundus exam. An independent, un-masked Data Safety Monitoring Board will meet
periodically to evaluate the study. Subject will be discontinued if, during the study, they
demonstrate a IVCM Score > baseline IVCM Score, or > IVCM Score at any previous visit.
Efficacy will be assessed at each clinic visit.
At each clinic visit, Study Staff will have/help the subject remove Ophthalmic Insert and
assessments will be conducted by Investigator masked to study treatment.

| | |
|---|---|
| Masking: Observer- | Randomization: AGENT Ophthalmic Insert versus Placebo Insert |
| masked. | (2:1) |
| Number of Subjects: 30 | Location and Number of Sites: up to 6, US, Europe and Australia |

Inclusion Criteria:
At Screening and Day 1 subjects must meet all of the following inclusion criteria:
1. Males or females, between 5 years [younger are less able to tolerate IVCM (Liang 2015)]
and 30 years of age, inclusive.
2. Diagnosis of ocular cystinosis.
3. Informed Consent or Caregiver Assent (pediatric subjects).
4. Baseline IVCM Score ≥2 (IVCM measured on a scale of 0 to 28).
5. Females will be nonpregnant and nonlactating, and females of childbearing potential and
males will agree to use contraception as detailed in the protocol.
6. Subject of their Caregiver must be able to comply with protocol.
Exclusion Criteria:
Subjects will be excluded from the study if they satisfy any of the following criteria at the
Screening visit unless otherwise stated:
1. History of hypersensitivity to sulfur or sulfite, NAC or any ingredient of the AGENT
Ophthalmic Insert.
2. Inability to wear daily AGENT Ophthalmic Insert. (Insert will be removed prior to clinical
assessments by an Investigator masked to study treatment.)
3. Contact lenses during the study.
4. Participation in a clinical study in the past 30 days, prior to Check-in.
5. Subjects who, in the opinion of the Investigator, should not participate in this study.
Investigational product, dosage
AGENT Ophthalmic Insert
Route of Administration: Topical ocular
Duration of the Study: 2 months
Criteria for Evaluation:
Safety: The safety assessments will include adverse events, BCVA, slit lamp biomicroscopy,
dilated fundus examination, limited physical exam, vital signs and clinical laboratory testing
of serum chemistry, hematology, urinalysis, and pregnancy test (for females of child-bearing
potential).
Efficacy: Efficacy will be assessed based on IVCM Score, and self- and investigator-assessed
photophobia. A decrease in IVCM total score from baseline indicates a reduction in corneal
crystals.
Safety Endpoints:
Incidence and severity of adverse events, as identified by physical examination, subject
reporting, clinically significant clinical laboratory abnormalities.

Study Plan

| | | Visit 2 | | | |
|---|---|---|---|---|---|
| | | Day 7 ± 3 days | Visit 3 | Visit 4 | Visit 5 |
| | Visit 1 | Telemedicine | Day | Day | Day |
| Study Procedures | Day 0 | Call | 30 ± 3 days | 60 ± 3 days | 90 ± 3 days |
| Informed consent | X | | | | |
| Inclusion/exclusion criteria | X | | | | |
| Demographic data | X | | | | |
| Medical history | X | | | | |
| Serum pregnancy test (females | X | | | | X |
| childbearing age) | | | | | |
| Study drug dispensing | X | X | X | X | X |
| Efficacy assessment (IVCM | X | | X | X | X |
| Score) | | | | | |
| Adverse event recording | X | X | X | X | X |
| Prior/concomitant medication | X | X | X | X | X |
| monitoring | | | | | |
| Clinical laboratory evaluations | X | | X | X | X |
| Vital signs, body temperature | X | | X | X | X |
| Ophthalmic examination (BCVA, | X | | X | X | X |
| slit lamp biomicroscopy, dilated | | | | | |
| fundus exam) | | | | | |
| Quality of Life Assessment | X | | | | X |
| Exit | | | | | X |

### Example 10

### Further Clinical Evaluation of an Ophthalmic Insert may Follow Example Phase 3 12-Month Clinical Study C-XX-XX of AGENT as per Ophthalmic Insert Synopsis Below (

AGENT: NACA, diNACA, NAC, cysteamine (or any salt form) or any cystine-depleting agent.

Name of Sponsor/Company: Nacuity Pharmaceuticals Inc.
Name of Investigational Product: AGENT Ophthalmic Insert
Name of Active Ingredient: AGENT
Title of Study: An Open-Label Phase 2/3 Study of the Safety, Efficacy and Pharmacokinet-
ics of AGENT Ophthalmic Insert for Treatment of Ocular Effects of Cystinosis
Study Number: C-XX-XX

| | |
|---|---|
| Study Period: 12 | Phase of Development: Phase 2/3 |
| months | |

Primary Objectives:
Evaluate the safety of AGENT Ophthalmic Insert in cystinosis subjects based on
BCVA, slit lamp biomicroscopy and dilated fundus exam.
Evaluate efficacy of AGENT Ophthalmic Insert based on comparison of IVCM Score*
at each visit versus baseline.
Exploratory Objectives:
Evaluate efficacy of AGENT Ophthalmic Insert based on comparison of the mean
density of inflammatory cells (IVCM-inf)** at each visit versus IVCM-inf at baseline.
Evaluate efficacy of AGENT Ophthalmic Insert based on comparison of AS-OCT** at
each visit versus AS-OCT at baseline.
Evaluate efficacy of AGENT Ophthalmic Insert based on comparison of the clinician-
assessed photophobia** at each visit versus baseline.
Evaluate efficacy of AGENT Ophthalmic Insert based on comparison of the subject-
assessed photophobia** at each visit versus baseline.
Evaluate pharmacokinetics (plasma AGENT) in selected subjects.
Evaluate the treatment acceptability via questionnaire for subjects treated with AGENT
Ophthalmic Insert at baseline versus Exit Visit.
(*Labbe et al., 2009; **Liang et al., 2015)
Methodology:
This open-label study will evaluate the safety, efficacy and pharmacokinetics of DRUG
Ophthalmic Insert for treatment of ocular effects of cystinosis in pediatric and adult
subjects.
Efficacy will be assessed based on IVCM score, AS-OCT, and self- and investigator-
assessed photophobia. Subjects must have a baseline IVCM score of ≥4. A decrease in
IVCM Score from baseline indicates a reduction in corneal crystals.
Safety will be assessed based on evaluation of adverse events, BCVA, slit lamp
biomicroscopy and dilated fundus exam. An independent, un-masked Data Safety Monitor-
ing Board will meet periodically to evaluate the study. Subject will be discontinued if,
during the study, they demonstrate a IVCM Score > baseline IVCM Score, or > IVCM
Score at any previous visit.
A Quality of Life questionnaire will be administered at Baseline and Exit Visits.
Pharmacokinetics of AGENT and major metabolite(s) in plasma will be assessed in selected
subjects (N = 6).
Interim report may be utilized for regulatory submission.

| | |
|---|---|
| Masking: Open- | Randomization: None |
| Label. | |
| Number of Subjects: | Location and Number of Sites: up to 6, US, Europe and Australia |
| 20 | |

Inclusion Criteria:
At Screening and Day 1 subjects must meet all of the following inclusion criteria:
1. Males or females between 1 year and 50 years of age, inclusive.
2. Diagnosis of ocular cystinosis.
3. Informed consent or Caregiver Assent (pediatric subjects).
4. Baseline IVCM Score ≥2 (on a scale of X to Y).
5. Females will be nonpregnant and nonlactating, and females of childbearing potential
and males will agree to use contraception as detailed in the protocol.
6. Subject (or their caregiver) must be able to comply with protocol.
Exclusion Criteria:
Subjects will be excluded from the study if they satisfy any of the following criteria at the
Screening visit unless otherwise stated:
1. History of hypersensitivity to sulfur or sulfite, NAC or any ingredient of the AGENT
Ophthalmic Insert.
2. Inability to wear daily AGENT Ophthalmic Insert. (Insert will be removed prior to
clinical assessment by Investigator masked to study treatment.)
3. Contact lenses during the study.
4. Participation in a clinical in the past 30 days, prior to Check-in.
6. Subjects who, in the opinion of the Investigator, should not participate in this study.
Investigational product, dosage
AGENT Ophthalmic Insert
Route of Administration: Topical ocular
Criteria for Evaluation:
Safety: The safety assessments will include adverse events, BCVA, slit lamp biomicroscopy,
dilated fundus examination, limited physical exam, vital signs and clinical laboratory testing
of serum chemistry, hematology, urinalysis, and pregnancy test (for women of child-bearing
potential).
Efficacy: Efficacy will be assessed based on IVCM Score, AS-OCT, and self- and PI-
assessed photophobia. A decrease in IVCM Score from baseline indicates a reduction in
corneal crystals.
Pharmacokinetics: AGENT and major metabolite(s) in plasma will be assessed in selected
subjects (N = 5).
Safety Endpoints:
Incidence and severity of adverse events, as identified by physical examination, subject
reporting, clinically significant clinical laboratory abnormalities, as well as BCVA, slit lamp
biomicroscopy and dilated fundus exam.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of' or “consisting of”. As used herein, the phrase” consisting essentially of requires the specified integer(s) or steps as well as those that do not materially affect the character or function of the claimed invention. As used herein, the term “consisting” issued to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. Each of the composition of the present invention may comprise NACA or diNACA, may consist essentially of NACA or diNACA, or may consist of NACA or diNACA, as outlined hereinabove.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refer condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organization cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Abderhalden E. Familiare cystindiathese. Z. Physiol. Chem. 1903;38:557.

Ariceta G, Giordano V, Santos F. Effects of long-term cysteamine treatment in patients with cystinosis. Pediatr Nephrol. 2019; 34(4):571 □578. doi:10.1007/s00467-017-3856-4.

Ates B, Abraham L, Ercal N. Antioxidant and free radical scavenging properties of N-acetylcysteine amide (NACA) and comparison with N-acetylcysteine (NAC). Free Radic Res. 2008; 42(4): 372-7.

Atlas D, Melamed E, Ofen D. Brain targeted low molecular weight hydrophobic antioxidant compounds [Internet]. U.S. Pat. No. 5,874,468 A 1999 accessed at: www. google. com/patents/U.S. Pat. No. 5,874,468.

Baranowski P, Karolewicz B, Gajda M, Pluta J. Ophthalmic drug dosage forms: characterisation and research methods. Scientific World Journal. 2014 Mar. 18; 2014:861904. doi: 10.1155/2014/861904. PMID: 24772038; PMCID: PMC3977496.

Baumner S and Weber L T (2018) Nephropathic Cystinosis: Symptoms, Treatment, and Perspectives of a Systemic Disease. Front. Pediatr. 6:58. doi: 10.3389/fped.2018.00058.

Bishop R J, Hohenfellner K, Lian H. Ophthalmology cystinosis forum morning session. EMJ Nephrol 2017; 5(54):2-9.

Broyer M, Tete M J, Gubler M C. Late symptoms in infantile cystinosis. Pediatr Nephrol. 1987; 1(3):519-24. doi: 10.1007/BF00849263. PMID: 3153326.

Buchan B, Kay G, Henegan A, Matthews KH, Cairns D. Gel formulations for treatment of the ophthalmic complications in cystinosis. Int. J. Pharmaceutics, 2010; 392(1-2); 192-197. doi: 10.1016/j.ijpharm.2010.03.065. Epub 2010 Apr 9.PMID: 20382212.

Buchan B, Kay G, Matthews K H, Cairns D. Suppository formulations as a potential treatment for nephropathic cystinosis. J Pharm Sci 2012; 101(10):3729-38. doi: 10.1002/jps.23246. Epub 2012 Jul. 6.PMID: 22778070.

Burki E. Uber die cystinkrankheit im kleinkindesalter unter besonderer berucksichtigung des augenbefundes. Ophthalmologica 1941; 101:257.

CYSTARAN® Prescribing Information, April 2020.

CYSTADROPS® Prescribing Information, accessed 2021 May 18 at: www.accessdata.fda.gov/.

David D, Princiero Berlingerio S, Elmonem M A, Oliveira Arcolino F, Soliman N, van den Heuvel B, Gijsbers R, Levtchenko E. Molecular Basis of Cystinosis: Geographic Distribution, Functional Consequences of Mutations in the CTNS Gene, and Potential for Repair. Nephron. 2019; 141(2):133-146. doi: 10.1159/000495270. Epub 2018 Dec. 14. PMID: 30554218.

Elmonem, et al., Orphanet Journal of Rare Diseases (2016) 11:47, page 13 of 17).

Emma F, Nesterova G, Langman C, Labbé A, Cherqui S, Goodyer P, Janssen M C, Greco M, Topaloglu R, Elenberg E, Dohil R, Trauner D, Antignac C, Cochat P, Kaskel F, Servais A, Wühl E, Niaudet P, Van't Hoff W, Gahl W, Levtchenko E. Nephropathic cystinosis: an international consensus document. Nephrol Dial Transplant. 2014; 29(Suppl 4):iv87-94. doi: 10.1093/ndt/gfu090. PMID: 25165189; PMCID: PMC4158338.

Genetic and Rare Disease Information Center Website accessed Jun. 12, 2020: rarediseases.info.nih.gov/diseases/6236/cystinosis.

Guimaraes L R D F, Seguro A C, Shimizu M H M, Neri L A L, Sumita N M, Breganca A C D, Volpini R A, Sanches T R C, Fonseca F A M D, Filbo C A M, Vaisbach M H. N-acetyl-cysteine is associated to renal function improvement in patients with nephropathic cystinosis. Pediatric Nephrol 2013 published online DOI 10.1007/s00467-013-2705-3.

Huynh N, Gahl W A Bishop R J. Cysteamine ophthalmic solution 0.44% for the treatment of corneal cystine crystals in cystinosis, Expert Review of Ophthalmology 2013; 8(4):341-345. DOI: 10.1586/17469899.2013.814885.

Kahns A H, Bundgaard H. Prodrugs as drug delivery systems. 107. Synthesis and chemical and enzymatic hydrolysis kinetics of various mono- and diester prodrugs of N-acetylcysteine. Int J Pharm. 1990; 62(2):193-205.

Kaiser-Kupfer M I, Chan C C, Rodrigues M, Datiles M B, Gahl W A. Nephropathic cystinosis: immunohistochemical and histopathologic studies of cornea, conjunctiva and iris. Curr Eye Res. 1987; 6(4):617-22. doi: 10.3109/02713688709025222. PMID: 3581880.

Kelly G S. Clinical applications of N-acetylcysteine. Altern Med Rev J Clin Ther. 1998; 3(2):114-27.

King B; Vance J; Wall G M; Shoup R. Quantitation of free and total N-acetylcysteine amide and its metabolite N-acetylcysteine in human plasma using derivatization and electrospray LC-MS/MS. J Chrom B 2019; 1109:25-36.

Labbé A, Niaudet P, Loirat C, Charbit M, Guest G, Baudouin C. In vivo confocal microscopy and anterior segment optical coherence tomography analysis of the cornea in nephropathic cystinosis. Ophthalmology. 2009 May; 116(5):870-6. doi: 10.1016/j.ophtha.2008.11.021. PMID: 19410944.

Liang H, Baudouin C, Tahiri Joutei Hassani R, Brignole-Baudouin F, Labbe A. Photophobia and corneal crystal density in nephropathic cystinosis: an in vivo confocal microscopy and anterior-segment optical coherence tomography study. Invest Ophthalmol Vis Sci. 2015 May; 56(5):3218-25. doi: 10.1167/iovs.15-16499. PMID: 26024106.

McCaughan B, Kay G, Knott R, Cairns D. A potential new prodrug for the treatment of cystinosis: design, synthesis and in-vitro evaluation. Bioorg Med Chem Letters 2008; 18(5):1716-9. doi: 10.1016/ibmc1.2008.01.039. Epub 2008 Jan. 18.

Neil J; Wall G M; Brown M. Antioxidant Effects of N-acetylcysteine Amide and NPI-002 on Human Skin or Equivalents. AAPS PharmSci360, Poster Abstract, October 26-Nov. 5, 2020.

Nesterova G and Gahl W A. Infantile nephropathic cystinosis: standards of care. Cystinosis Research Network. 2012. Accessed 2021 May 19 at: cystinosis.org/wp-content/uploads/2019/01/CRN_StandardsOfCare FINAL-1.pdf

Omran Z, Maloney KA, Benylles A, Kay G, Knott R, Cairns D. Synthesis and in vitro evaluation of novel pro-drugs for the treatment of nephropathic cystinosis. Bioorg. Med. Chem. Lett. 2011a; 19:3492-3496.

Omran Z, Kay G, Slvo A D, Knott R, Cairns D. PEGylated derivatives of cystamine as enhanced treatments for nephropathic cystinosis. Bioorg Med Chem Letters 2011b; 21:45-47.

Omran Z, Kay G, Hector E E, Knott R M, Cairns D. Folate pro-drug of cystamine as an enhanced treatment for nephropathic cystinosis. Bioorg Med Chem Lett. 2011c; 21(8):2502-4. doi: 10.1016/ibmc1.2011.02.048. Epub 2011 Feb. 23. PMID: 21397500.

Pescina S, Carra F, Padula C, Santi P, Nicoli S. Effect of pH and penetration enhancers on cysteamine stability and trans-corneal transport. Eur J Pharm Biopharm. 2016; 107:171-9. doi: 10.1016/j.ejpb.2016.07.009. Epub 2016 Jul. 6. PMID: 27395395.

Pinxten A M, Hua M T, Simpson J, Hohenfellner K, Levtchenko E, Casteels I. Clinical Practice: A Proposed Standardized Ophthalmological Assessment for Patients with Cystinosis. Ophthalmol Ther. 2017; 6(1):93-104. doi: 10.1007/s40123-017-0089-3. Epub 2017 May 5. PMID: 28477325; PMCID: PMC5449308.

Rathore K S and Nema R K. Review on ocular inserts. Int J PharmTech Res. 2009; 1(2): 164-169.

Shams F, Livingstone I, Oladiwura D, Ramaesh K. Treatment of corneal cystine crystal accumulation in patients with cystinosis. Clin Ophthalmol. 2014; 8:2077-84. doi: 10.2147/OPTH.S36626. PMID: 25336909; PMCID: PMC4199850.

Summary Basis of Approval NDA200740 accessed Jul. 7, 2020 at: www.accessdata.fda.gov/drugsatfda_docs/nda/2012/2007400rig1s000SumR.pdf.

Sunitha K, Hemshekhar M, Thushara R M, Santhosh M S, Yariswamy M, Kemparaju K, Girish K S. N-Acetylcysteine amide: a derivative to fulfill the promises of N-Acetylcysteine. Free Radic Res. 2013 May; 47(5):357-67. doi: 10.3109/10715762.2013.781595. Epub 2013 Apr. 8. PMID: 23472882.

Tsilou E T, Rubin B I, Reed G, Caruso R C, Iwata F, Balog J, Gahl W A, Kaiser-Kupfer M I. Nephropathic cystinosis: posterior segment manifestations and effects of cysteamine therapy. Ophthalmology. 2006; 113(6):1002-9. doi: 10.1016/j.ophtha.2005.12.026. Epub 2006 Apr. 17. PMID: 16603246. Tsilou E T, Rubin B I, Reed G F, Iwata F, Gahl W, Kaiser-Kupfer M I. Age-related prevalence of anterior segment complications in patients with infantile nephropathic cystinosis. Cornea. 2002; 21(2):173-6. doi: 10.1097/00003226-200203000-00009. PMID: 11862089.

Vaisbich M H, Pache de Faria Guimaraes L, Shimizu M H, Seguro A C. Oxidative stress in cystinosis patients. Nephron Extra. 2011 January; 1(1):73-7. doi: 10.1159/000331445. Epub 2011 Sep. 19. PMID: 22470381; PMCID: PMC3290844.

Wong V G, Lietman P S, Seegmiller J E. Alterations of pigment epithelium in cystinosis. Arch Ophthalmol. 1967; 77(3):361-9. doi: 10.1001/archopht.1967.00980020363014. PMID: 5297572.

What is claimed is:

1. A method for treatment of cystinosis in a human subject that comprises:
administering to the human patient a therapeutically effective amount of at least one of:
N-acetylcysteine amide (NACA) or (2R,2R′)-3,3′-disulfanediyl bis(2-acetamidopropanamide) (diNACA).

2. The method of claim 1, wherein the NACA or diNACA is provided in or with a pharmaceutically acceptable carrier.

3. The method of claim 1, wherein the NACA or diNACA is administered orally, intravenously, intramuscularly, enterally, intraocularly, subretinally, intravitreally, posterior juxtasclerally, anterior juxtasclerally, retrobulbarly, topically, topical ocularly, ophthalmically or with an ocular insert, sublingually, or rectally.

4. The method of claim 1 wherein the NACA or diNACA is administered in a bio erodible or nonbioerodible ophthalmic or ocular insert.

5. The method of claim 1 wherein the NACA or diNACA is administered in a bio erodible ophthalmic or ocular insert once or twice daily.

6. The method of claim 1 wherein the NACA or diNACA is administered in a nonbiodegradable ophthalmic or ocular insert that is changed once or twice daily.

7. The method of claim 1, wherein the NACA or diNACA is administered in daily doses of about 0.01 to 150 mg/Kg.

8. The method of claim 1, wherein the NACA or diNACA is administered two or three times daily.

9. The method of claim 1, wherein the NACA or diNACA is administered once per day.

10. The method of claim 1, wherein the NACA or diNACA is administered with a second active agent.

11. The method of claim 1, wherein the NACA or diNACA is administered with a second active agent selected from at least one of cysteamine (or any salt form), N-acetylcysteine, ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, α-tocopherol, citric acid, ethylenediamine tetraacetic acid (EDTA), sorbitol, tartaric acid, or phosphoric acid.

12. The method of claim 1, wherein the dose for administration is 100, 150, 300, 333, 400, 500, 600, 700, 750, 800, 900, 1,000, 2,500, 5,000, 7,500, or 10,000 micrograms per dose.

13. The method of claim 1, wherein the dose for administration is 100, 150, 300, 333, 400, 500, 600, 700, 750, 800, 900, 1,000, 2,500, 5,000, 7,500, or 10,000 mg per dose.

14. The method of claim 1, wherein the NACA or diNACA is delivered orally via a mini-tablet, tablet, capsule, tablet, effervescent, dual release, mixed release, sachet, powder or liquid.

15. The method of claim 1, wherein the NACA or diNACA is administered to prevent, given prophylactically, or to treat cystinosis.

16. The method of claim 1, wherein the NACA or diNACA are administered to reduce cystine levels anywhere in the body of a person afflicted with cystinosis.

17. A method for the treatment of cystinosis comprising:
identifying a human in need of treatment for cystinosis; and
administering to the human a therapeutically effective amount of a cystine-depleting agent in an ophthalmic or ocular insert sufficient to treat ophthalmic, ocular or corneal cystinosis.

18. A method for the treatment of cystinosis comprising:
identifying a human in need of treatment for cystinosis; and
administering to the human a therapeutically effective amount of a NAC or cysteamine (or any salt form) in an ophthalmic or ocular insert sufficient to treat ophthalmic, ocular or corneal cystinosis.

19. The method of claim 18 wherein the NAC or cysteamine (or any salt form) is administered in a bioerodible or nonbioerodible ophthalmic or ocular insert.

20. The method of claim 18 wherein the NAC or cysteamine (or a salt thereof) is administered in a bio erodible ophthalmic or ocular insert once or twice daily.

21. The method of claim 18 wherein the NAC or cysteamine (or a salt thereof) is administered in a nonbiodegradable ophthalmic or ocular insert that is changed once or twice daily.

22. The method of claim 18, wherein the NAC or cysteamine (or a salt thereof) is administered in daily doses of about 0.01 to 150 mg/Kg.

23. The method of claim 18, wherein the NAC or cysteamine (or a salt thereof) is administered two or three times daily.

24. The method of claim 18, wherein the NAC or cysteamine (or a salt thereof) is administered once per day.

25. A method for treatment of cystinosis in a human subject that comprises:
identifying a human patient in need of treatment cystinosis; and
administering to the human patient a therapeutically effective amount of at least one of:
N-acetylcysteine amide (NACA) or (2R,2R′)-3,3′-disulfanediyl bis(2-acetamidopropanamide) (diNACA).

26. The method of claim 25, wherein the NACA or diNACA is administered with a second active agent selected from at least one of: cysteamine, N-acetylcysteine, cysteine hydrochloride, or other cystine-depleting agent.
